(19) United States
(12) Patent Application Publication
(10) Pub. No.: US 2023/0021892 A1
(43) Pub. Date: Jan. 26, 2023

(54) UNIT FOR A MECHANICAL CONNECTING DEVICE FOR MEDICAL PURPOSES, PARTICULARLY FOR PERITONEAL DIALYSIS

(71) Applicant: PERIPAL AG, Zürich (CH)

(72) Inventors: Sandra NEUMANN, Zurich (CH); Jean-Claude GRÖBLI, Greifensee (CH)

(73) Assignee: PERIPAL AG, Zurich (CH)

(21) Appl. No.: 17/256,017

(22) PCT Filed: Jun. 25, 2019
(86) PCT No.: PCT/EP2019/066889

§ 371 Date: Dec. 24, 2020

(30) Foreign Application Priority Data
Jun. 25, 2018 (EP) 18179458.7

(51) Int. Cl. A61M 39/16; A61M 1/28; A61M 39/10

(52) U.S. Cl. CPC A61M 39/165; A61M 39/162; A61M 1/28; A61M 39/1011; A61M 2039/087

(57) ABSTRACT

The invention relates to a unit (1), particularly for use with an apparatus (2) configured to connect a tubular fitting (24) to a connector (14) and to disconnect the tubular fitting (24) from the connector (14), the unit (1) comprising: a body (16) having a first receptacle (18) and a connector holder (26) configured to hold the connector (14), a second receptacle (20), wherein each of said receptacles (18, 20) is configured for holding an end cap (22) of the tubular fitting (24), and wherein the unit (1) further comprises a capsule (290) that is configured to be connected to said body (16) in a releasable fashion, wherein said second receptacle (20) is formed by the capsule (290). Furthermore, the invention relates to a capsule (290) and to an apparatus (2).

The present disclosure generally relates to an (e.g. disposable) capsule of a unit of an apparatus for connecting and disconnecting a tubular fitting to a connector for medical purposes, particularly for peritoneal dialysis, and more specifically to an (e.g. disposable) capsule of a unit for an apparatus configured to replace an end cap of a medical catheter in a protective and sterile environment. The invention further relates to the unit and to the apparatus.

Connections of tubular fittings for active fluidic delivery are often achieved by Luer-lock connecting systems. Often, these conventional Luer-lock connecting systems are used to perform peritoneal dialysis and the like by employing an actuating station having a pumping mechanism and associated flexible tubes or using manual therapy approaches. A user connects and disconnects a catheter (or similar tubing, i.e. transfer set) from the user to the connecting system at least once a day, or up to five times a day for exchanging medical fluid to and from the user's peritoneal cavity. To initiate the peritoneal dialysis, an old, existing end cap of the catheter is initially disconnected from the catheter, and a connection is made between the catheter and a dialysate fluid bag, or a tubing leading to a fluid or a drug delivery system (e.g. pump). For example, in the state of the art, a stability barrier of the fluid bag is removed, and the catheter is rotationally fastened to an end tube of the fluid bag. After completion of the dialysis, the user rotationally unfastens the catheter from the fluid bag and reseals the catheter with a new, sterile end cap.

However, during exchanges of the catheter end caps from an old one to a new one, the catheter and the fluid bag connection are typically exposed to an unsterile environment. At times, peritonitis can occur due to contamination caused by contact with the surrounding atmosphere, and accidental touching of the catheter.

Other catheter related infections can also occur due to touch or air (e.g. breath) contact during the connection and disconnection of the connecting system. This situation is undesirable to the user because bacteria and other micro-organisms can be introduced into the user's peritoneal cavity, thereby causing peritonitis or other illness. Moreover, the conventional system relies in part on the vision and/or skills of the user for properly connecting and disconnecting the catheter.

Thus, the problem underlying the present invention is to provide a device that allows replacement or interchange of end caps in a simple and sanitary manner.

This problem is solved by a unit having the features of claim 1. Preferred embodiments of the unit according to the invention are stated in the corresponding sub claims and are described below. Further aspects of the present invention relate to an apparatus comprising a unit according to the present invention, as well as to a capsule containing an end cap.

According to claim 1 a unit, particularly a disposable unit, is disclosed, wherein particularly the unit is configured for use with an apparatus configured to connect a tubular fitting to a connector and to disconnect the tubular fitting from the connector, and/or wherein particularly the unit is configured to be connected to the apparatus, wherein the unit comprises a body comprising a first receptacle, and wherein the unit further comprises a second receptacle, wherein particularly said first and said second receptacles can be disposed or can be disposable at opposite ends of said body, and wherein each of said receptacles is configured to receive or hold an end cap of the tubular fitting, particularly to fasten or unfasten the end cap for replacement. The body further comprises a connector holder that can be disposed between said first and second receptacles and is configured to hold the connector. Preferably, in an embodiment, the body is configured to be connected to said apparatus in a releasable fashion, particularly such that the unit can be manually connected to said apparatus and manually removed from the apparatus, e.g. after its use (see below).

Further, the unit comprises a capsule that is configured to be connected to said body in an (e.g. manually) releasable fashion, wherein said capsule forms said second receptacle.

Particularly, the position of the first and second receptacle may also be interchanged, i.e., when looking from above, the first receptacle may be arranged on the left side of the connector holder or on the right side of the connector holder. The movements of the movable carrier/unit can be easily adapted to such a change of positions.

Particularly, the first receptacle does not have to enclose an end cap received therein completely. It suffices when at least a portion of the end cap can be arranged or received in the receptacle to hold the end cap. Thus, the notion that the end cap is received in the first receptacle also means that at least a portion of the end cap is received or arranged in the first receptacle. Particularly, this allows to hold the end cap using the first receptacle.

According to an embodiment, the capsule is configured to be sterilized for allowing multiple uses. Alternatively, the capsule is a disposable capsule that is designed for a single use within the unit, particularly comprising unfastening an end cap from the tubular fitting, connecting the connector inserted into the connector holder to the tubular fitting, disconnecting the connector from the tubular fitting, and fastening an end cap received in the second receptacle (e.g. capsule) to the tubular fitting.

Further, according to an embodiment of the unit according to the present invention, the body comprises a recess configured to receive the capsule, preferably in a form fitting manner.

Further, according to an embodiment of the unit, the recess of the body for receiving the capsule is delimited by a lateral wall configured to enclose the capsule at least partially when the capsule is arranged in said recess of the body. Particularly, said lateral wall delimiting the recess of the body can comprise a gap so that said lateral wall comprises two (e.g. separate) sections separated by said gap.

Further, according to an embodiment of the unit, the capsule comprises an internal space for receiving an end cap. The internal space may therefore also be denoted as second receptacle since it receives the end cap. Particularly, the end cap is arranged in the internal space of the capsule.

Further, according to an embodiment of the unit, the lateral wall of the capsule surrounds an internal space of the capsule in a peripheral direction of the capsule, wherein particularly the lateral wall can be cylindrical or can comprise a cylindrical portion. Particularly, in an embodiment, the lateral wall of the capsule comprises an outside facing away from the internal space of the capsule. Furthermore, in an embodiment, the lateral wall of the capsule comprises an inside facing the end cap residing in the internal space of the capsule, wherein particularly said outside forms a cylindrical surface.

Particularly, in an embodiment of the unit, said lateral wall of the recess of the body is configured to support the lateral wall of the capsule when the capsule is arranged in the recess of the body and rests on the lateral wall of the recess of the body.

Further, according to an embodiment of the unit, the capsule comprises a bottom wall connected to the lateral wall of the capsule, wherein the lateral wall of the capsule extends from the bottom wall in an axial direction of the capsule towards a face side of the lateral wall of the capsule, wherein the face side faces away from an outside of the bottom wall of the capsule. Particularly, the bottom wall of the capsule can be circular.

Further, according to an embodiment of the unit, the capsule comprises an axial protrusion protruding in the axial direction from the bottom wall of the capsule on the outside of the bottom wall, wherein the axial protrusion is configured to engage with a portion of said recess of the body, particularly in a form fitting manner, when the capsule is arranged in the recess of the body of the unit.

Further, according to an embodiment of the unit, the axial protrusion is configured to engage with said portion of the recess of the body upon arranging the capsule in the recess of the body so that a self-positioning of the capsule with respect to the peripheral direction of the capsule occurs.

Further, according to an embodiment of the unit, the axial protrusion tapers, particularly in a direction perpendicular to the axial direction of the capsule, and wherein said portion of the recess of the body is formed as a slot that tapers, which slot is particularly configured to receive the axial protrusion of the capsule in a form fitting manner.

Further, according to an embodiment of the unit, the axial protrusion is wedge-shaped, particularly tapering in a direction perpendicular to the axial direction, and wherein particularly said portion of the recess of the body forms a wedge-shaped slot.

Further, according to an alternative embodiment of the unit, the axial protrusion comprises a cylindrical shape (or comprises at least a cylindrical portion), and wherein particularly said portion of the recess of the body forms a slot, particularly a slot with a round end, wherein this slot is configured to receive the axial protrusion.

Further, according to an embodiment of the unit, the unit comprises an end cap arranged in the internal space formed by the capsule, wherein particularly said end cap is completely arranged in said internal space.

Further, according to an embodiment of the unit, the end cap comprises a protrusion having a basis and a head, wherein particularly the head forms a free end of the protrusion of the end cap, and wherein particularly the head comprises a diameter perpendicular to a direction in which the protrusion of the end cap protrudes from the end cap, wherein this diameter is larger than a diameter of the basis of the protrusion of the end cap perpendicular to said direction. Further, particularly, the head is configured to engage behind an edge of the first receptacle of the unit, so that the tubular fitting can be pulled off the end cap when the end cap has its head engaged with said edge of the first receptacle.

Further, according to an embodiment of the unit, said axial protrusion surrounds a portion of the internal space of the capsule, wherein said protrusion of the end cap is arranged in said portion of the internal space so that particularly the end cap residing in the internal space comprises a fixed position (e.g. with respect to the lateral wall of the capsule).

Further, according to an embodiment of the unit, the capsule comprises a lateral protrusion protruding (particularly in a radial direction of the capsule) from the lateral wall on an outside of the lateral wall, wherein the lateral protrusion extends in the peripheral direction of the capsule. Further, particularly, the respective radial direction runs perpendicular to the axial direction of the capsule.

Further, according to an embodiment of the unit, the lateral protrusion is spaced apart from the face side of the lateral wall in the axial direction of the capsule.

Further, according to an embodiment of the unit, the lateral protrusion is spaced apart from the outside of the bottom wall in the axial direction of the capsule.

Further, according to an embodiment of the unit, the lateral protrusion is arranged adjacent the bottom wall, particularly adjacent the outside of the bottom wall of the capsule.

Further, according to an embodiment of the unit, the lateral protrusion is arranged adjacent the face side of the lateral wall.

Further, according to an embodiment of the unit, the lateral protrusion is a circumferential lateral protrusion.

Further, according to an embodiment of the unit, the lateral protrusion comprises at least one gap.

Further, according to an embodiment of the unit, the lateral protrusion comprises a first gap and a second gap.

Further, according to an embodiment of the unit, the lateral protrusion comprises a third gap, wherein particularly the third gap branches off the first gap in the peripheral direction of the capsule or is adjacent to the first gap.

Further, according to an embodiment of the unit, the lateral protrusion comprises a first radial projection adjacent to the first gap.

Further, according to an embodiment of the unit, the lateral protrusion comprises a second radial projection adjacent to the first gap, wherein particularly the second radial projection is arranged adjacent the third gap, wherein particularly the second radial projection is shaped as an arrow pointing away from the capsule, particularly in a radial direction of the capsule.

Further, according to an embodiment of the unit, a portion of the lateral wall delimiting the recess of the body of the unit is configured to engage into the gap or into the first gap and/or the second gap of the lateral protrusion when the capsule is arranged in said recess of the body of the unit.

In particular, the portion of the lateral wall is configured to engage into the third gap, particularly configured to automatically engage into the third gap, for removing the capsule from the unit. In particular, this insertion or engagement of the portion of the lateral wall into the third gap may take place when the fastening lever of the unit is moved from the second position to the first position while the capsule is connected to the fastening lever.

Particularly, when the lateral wall of the recess of the body of the unit comprises two sections separated by the gap (as described above) a portion of each section of the lateral wall is configured to engage with the gap of the lateral protrusion of the lateral wall of the capsule.

Further, according to an embodiment of the unit, the lateral protrusion of the capsule is configured to engage with a groove formed in the body of the unit. Particularly, this groove is formed in the lateral wall of the recess of the body.

Further, according to an embodiment of the unit, the capsule comprises a further lateral protrusion that protrudes from the lateral wall of the capsule on the outside of the lateral wall of the capsule, wherein the further lateral protrusion extends in the peripheral direction of the capsule.

Further, according to an embodiment of the unit, the further lateral protrusion is spaced apart from said lateral protrusion in the axial direction of the capsule and particularly faces said lateral protrusion in the axial direction of the capsule.

Further, according to an embodiment of the unit, the further lateral protrusion is arranged adjacent the face side of the lateral wall of the capsule. Particularly, the further lateral protrusion can be flush with the face side of the lateral wall of the capsule. Further, particularly, the face side delimits an opening of the capsule via which the internal space of the capsule is accessible, wherein said opening is closed (and covered) in an embodiment by a closure, see e.g. also below.

Further, according to an embodiment of the unit, the further lateral protrusion is a circumferential lateral protrusion.

Further, according to an embodiment of the unit, the capsule comprises a closure configured to close said internal space of the capsule, particularly hermetically, wherein said closure is attached to the circumferential face side of the lateral wall of the capsule (and particularly to the further lateral protrusion) of the lateral wall of the capsule. Particularly, the closure can be attached to the face side and/or to the further lateral protrusion of the lateral wall of the capsule by heating the closure and pressing the (e.g. partially molten) closure onto the face side and/or further lateral protrusion of the lateral wall of the capsule so that the closure sticks to the lateral wall after curing of the closure. The closure may also be glued to the lateral wall by means of a glue. Other ways of connecting the closure to the lateral wall of the capsule are also possible.

Further, according to an embodiment of the unit, the closure is or comprises a peel-off seal (e.g. a seal that is configured to be peeled off the face side and/or further lateral protrusion of the lateral wall of the capsule).

Further, according to an embodiment of the unit, the closure comprises at least one through-opening, wherein particularly the at least one through-opening can be one of: an oblong hole, a quadrangular hole, a square hole, a circular hole. Particularly, according to an embodiment of the unit, the at least one through-opening is formed in an (e.g. flexible) edge region of the closure, wherein particularly said edge region is a free edge region, and wherein particularly the edge region protrudes from the lateral wall of the capsule perpendicular to the axial direction of the capsule.

Further, according to an embodiment of the unit, the closure comprises a further through-opening, wherein particularly the further through-opening can be one of: an oblong hole, a quadrangular hole, a square hole, a circular hole. Further, in an embodiment, the further through-opening is formed in said edge region of the closure.

Further, according to an embodiment of the unit, said edge region comprises a section of an edge of the closure, which section extends linearly, wherein the at least one through-opening is arranged adjacent said section of the edge of the closure, and wherein particularly said further through-opening is arranged adjacent said section of the edge of the closure, too. Particularly, in an embodiment, the two through-openings are spaced apart from each other in a direction extending parallel to said section of the edge of the closure. Furthermore, particularly, the two through-openings are equidistantly spaced apart from said section of the edge of the closure in a direction perpendicular to said section of the edge of the closure.

Particularly, in an embodiment of the unit, the at least one through-opening, is configured to receive a hook element (particularly a hook element of an apparatus to which the unit is connected or connectable) to remove the closure, particularly to peel off, the closure (e.g. peel-off seal). Furthermore, in an embodiment, the further through-opening is configured to receive a further hook element of said apparatus to remove the closure, particularly to peel off the closure (e.g. peel-off seal).

Further, according to an embodiment of the unit, the closure comprises a first slot adjacent to the through-opening and a second slot adjacent to the further through-opening, wherein the through-opening and the further through-opening are arranged between the first slot and the second slot in the peripheral direction, and wherein particularly the first slot and the second slot extend perpendicular to the section of the edge of the closure.

Further, according to an alternative embodiment of the unit, said closure comprises a flexible strip connected to the peel-off seal, which strip comprises a free end section that forms a handle by means of which the peel-off seal can be peeled off the lateral wall of the capsule, particularly from outside or inside a housing of the apparatus, wherein particularly said free end section of the strip is configured to be arranged between a bottom and a cover of said housing such that the free end section of the strip protrudes out of the housing. Particularly, in an embodiment of the unit, the free end section of the strip is configured to be arranged between a lateral wall of the housing, which lateral wall is connected to the bottom of the housing, and the (e.g. pivotable) cover of the housing.

This is particularly useful for CAPD. However said end section (e.g. in case of APD) or handle can also be arranged in the housing and may be accessed after the housing has been opened.

Further, according to an embodiment of the unit, the unit comprises a pivotable fastening lever for fastening the capsule to the body in case the capsule is arranged in said recess of the body of the unit, wherein the fastening lever is mounted to the body of the unit. Particularly the fastening lever is configured to be pivoted manually.

Further, according to an embodiment of the unit, the fastening lever is pivotable between a first and a second position, wherein when the fastening lever is in the first position, the fastening lever releases the capsule, and wherein when the fastening lever is in the second position, the fastening lever fastens the capsule to the body of the unit in case the capsule is arranged in the recess of the body of the unit. Further, according to an embodiment of the unit, in the second position of the fastening lever, when the capsule is arranged in the recess of the body of the unit, a portion of the capsule is arranged between the fastening lever and the body to fasten the capsule to the body, particularly between the fastening lever and the lateral wall of the recess of the body.

Further, according to an embodiment of the unit, the fastening lever comprises a curved portion, wherein the curved portion comprises a concave inner surface, wherein the concave inner surface is configured to engage the lateral wall of the capsule in the second position.

Further, according to an embodiment of the unit, when the fastening lever is in the second position and the capsule is arranged in the recess of the body, the lateral protrusion of the capsule is configured to engage with the fastening lever, particularly with an end portion of the fastening lever.

Further, according to an embodiment of the unit, the fastening lever and/or the body, particularly at least one portion of the lateral wall of the body, is configured to rotate the capsule, particularly counter clockwise, when the fastening lever is moved from the first position to the second position. In particular, this improves positioning of the capsule in the recess of the body and/or facilitates insertion or engagement of the capsules into the recess of the body.

Further, according to an embodiment of the unit, the fastening lever comprises an extension configured to engage the lateral protrusion and/or the lateral wall of the capsule in the second position when the capsule is arranged in the recess of the body, wherein particularly the extension is configured to engage into the second gap of the lateral protrusion of the capsule. In particular, the extension extends in the axial direction of the capsule.

Further, according to an embodiment of the unit, when the fastening lever is in the second position and the capsule is arranged in the recess of the body, the fastening lever (e.g. said end portion of the fastening lever) is configured to lock the axial protrusion of the capsule that is arranged in said portion of the recess of the body (e.g. by covering an open side of said portion of the recess of the body or by engaging into the portion (e.g. slot) of the recess of the body of the unit perpendicular to said axial direction of the axial protrusion of the capsule.

Further, according to an embodiment of the unit, the fastening lever comprises a connecting means, particularly a slit or a recess, for connecting the fastening lever to the capsule. In particular, the slit or recess is configured to receive a part of the lateral protrusion of the capsule, more particularly in a form fitting manner. In particular, the slit or recess extends along the peripheral direction.

Further, according to an embodiment of the present invention, the capsule and/or the body are configured to fasten the capsule to the body of the unit by a latching connection when the capsule is arranged in said recess of the body adapted to receive the capsule.

Further, according to an embodiment of the present invention, said lateral wall of the recess of the body for enclosing the capsule at least partially when the capsule is arranged in said recess comprises a discontinuity arranged between two opposing edges of the lateral wall of the recess of the body.

Further, according to an embodiment of the present invention, a recess is formed in each edge, wherein these recesses formed in the edges face each other. Particularly, these recesses of said edges allow a more easy removal of the capsule from the recess of the body configured to receive the capsule.

Further, according to another embodiment of the present invention, an actuating member is arranged at each edge for unlocking the latching connection between the capsule and the recess or lateral wall of the recess. The actuating members may interact with one or several members of the latching connection between the capsule and the body in order to release the latching connection.

Further, according to an embodiment of the present invention, the lateral wall of the capsule comprises at least one latching nose that is configured to engage with an associated latching recess formed in said lateral wall of said recess for receiving the capsule, or wherein the lateral wall of said recess for receiving the capsule comprises at least one latching nose that is configured to engage with an associated latching recess formed in said lateral wall of the capsule.

Further, according to an embodiment of the present invention, the capsule comprises an indicator that is configured to indicate whether the capsule is unused (e.g. comprises a fresh end cap). Particularly, said indicator is arranged on the lateral wall of the capsule and can be a window or an indicator that undergoes a visually perceivable change when the capsule has been used (i.e. opened for using the end cap stored in the capsule).

Further, according to an embodiment of the present invention, the unit is a removable unit, wherein said body is configured to be connected to said apparatus in a releasable fashion (see also above).

Preferably, according to an embodiment, said body comprises a latching means or latching part for connecting the body in a releasable fashion to said apparatus.

Preferably, according to an embodiment, the unit is designed to be sterilized or the like for allowing multiple uses of the unit.

Preferably, according to an alternative embodiment, the unit is a disposable unit and particularly designed for a single use (and to be discarded thereafter) comprising at least: unfastening an end cap from the tubular fitting, connecting the connector inserted into the connector holder to the tubular fitting, disconnecting the connector from the tubular fitting, and fastening an end cap arranged in the capsule to the tubular fitting. Thus, the capsule and the whole unit can be disposable items.

Particularly, while the capsule is preferably adapted for a single use only, the unit may be designed for a finite number of such uses. Here, particularly, the removable unit is to be discarded before the end of the lifetime of the apparatus and to be substituted by a new removable unit.

Further, particularly, the connector (e.g. a so called Y-set forming part of the dialysis system) comprises a first conduit, which first conduit comprises a frangible inline seal, and a second conduit, wherein the two conduits branch off from an end section of the connector, which end section is configured to be connected to the tubular fitting (which e.g. forms part of or is connected to a catheter/transfer set) or disconnected from the tubular fitting, and wherein the tubular fitting comprises a member which is configured to be actuated so as to open or close the tubular fitting (or catheter) for allowing or preventing the passage of fluid through the tubular fitting.

In this regard, a single use of the disposable device can comprise the following steps (e.g. in case of CAPD):
providing the tubular fitting with an end cap fastened to the tubular fitting, wherein the end cap is received/held in the first receptacle of the body of the unit (wherein the unit is connected to a moveable carrier of the apparatus), and wherein the tubular fitting is inserted into the holder assembly,
particularly removing a closure (e.g. peel-off seal) of the capsule (which capsule is e.g. inserted into the recess of the body of the unit) to open the capsule which contains an end cap (removing the closure automatically can also be performed later on),
moving the movable carrier/unit away from the holder assembly so as to unfasten the end cap from the tubular fitting,
moving the movable carrier into the second position and then towards the holder assembly so as to connect the connector inserted into the connector holder to the tubular fitting,
preferably opening the tubular fitting (e.g. by allowing the patient or user to actuate said member on the tubular fitting) so as to drain a liquid or fluid, and closing the tubular fitting thereafter (e.g. using said member),
breaking the frangible inline seal using a first actuating member of the apparatus (see below),
flushing both conduits by letting fluid flow through the first and the second conduit while the tubular fitting is closed for preventing the passage of fluid through the tubular fitting,
preferably opening the tubular fitting (e.g. using said member, e.g. by allowing the patient or user to actuate said member on the tubular fitting) to let liquid or fluid flow (e.g. towards the target) before closing the tubular fitting (e.g. using said member again),
interrupting the second conduit by actuating a third actuating member of the apparatus and opening the tubular fitting (e.g. by actuating said member of the tubular fitting),
preferably letting fluid pass through the first conduit, the end section of the connector, and the tubular fitting towards a target,
closing the tubular fitting (e.g. by actuating said member of the tubular fitting),
interrupting the first conduit for preventing the passage of fluid through the first conduit by actuating the second actuating member,
closing the tubular fitting (e.g. by using said member) for preventing the passage of fluid through the tubular fitting,
particularly preventing any disconnection of the liquid of fluid line or between the tubular fitting and the connector before the tubular fitting is not closed (e.g. by an preferably mechanical detecting means that is configured to detect the opening/closing status) and particularly preventing retraction (e.g. of the tubular fitting) into the housing in case the tubular fitting is open. (Said detecting means may also be a mechanical means that is configured to prevent said retraction when the tubular fitting is open and allows the passage of fluid through the tubular fitting),
moving the movable carrier away from the holder assembly so as to disconnect the connector from the tubular fitting (here the closure may be removed automatically), and
moving the movable carrier into the third position and towards the holder assembly so as to fasten said end cap arranged in the capsule to the tubular fitting.

Particularly, the above described use corresponds to a CAPD, wherein particularly, in case of an APD said steps can also be conducted with the difference that the first, the second and the third actuating members are not used and particularly only a single conduit of the connector is present that leads to a cycler (which single conduit is arranged in the second region of the recess, while the first region stays empty):
providing the tubular fitting with an end cap fastened to the tubular fitting, wherein the end cap is received/held in the first receptacle of the body of the unit (the unit being connected to the movable carrier of the apparatus), wherein the movable carrier/unit resides in a first position, and wherein the tubular fitting is inserted into the holder assembly,
moving the movable carrier/unit away from the holder assembly so as to unfasten the end cap from the tubular fitting,
moving the movable carrier/unit into the second position and towards the holder assembly so as to connect the connector (comprising a single conduit) inserted into the connector holder to the tubular fitting,
preferably adding a removable clamp from above to arrest the connection between the tubular fitting and the connector while the tubular fitting is arranged in the holder assembly and the connector is arranged in the connector holder (e.g. recess of removable unit),
removing the tubular fitting and the connector connected thereto from the apparatus (and particularly from the unit),
opening the tubular fitting (e.g. by actuating said member of the tubular fitting) for letting fluid pass through the tubular fitting/connector,
closing the tubular fitting (e.g. by actuating said member of the tubular fitting),
reinserting the tubular fitting and the connector into the apparatus/unit,
preferably removing said removable clamp,
particularly removing a closure (e.g. peel-off seal) of the capsule to open the capsule which contains an end cap (this can also be done later on when the closure is removed automatically), and
moving the movable carrier/unit away from the holder assembly so as to disconnect the connector from the tubular fitting (here the closure may be automatically removed from the capsule) and optionally so as to move said member of the tubular fitting back into the housing,
moving the movable carrier/unit into the third position and towards the holder assembly so as to fasten said end cap arranged in the capsule to the tubular fitting.

After these complete cycles (e.g. APD or CAPD), the capsule (and particularly unit) can be discarded (e.g. together with the inserted connector and tubings connected thereto) and a new capsule can be inserted into the unit of the apparatus for the next use cycle.

According to a preferred embodiment of the unit according to the present invention, the (e.g. removable) unit comprises an end cap arranged in the second receptacle/internal space formed by the capsule, wherein said end cap is completely arranged in said internal space of the capsule. Said end cap can comprise a material and/or a Shore hardness as specified below.

Further, according to a preferred embodiment of the unit according to the present invention, a flexible means or flexible carrier member, particularly a sponge, that carries (e.g. has absorbed) an anti-bacterial fluid, is in contact with said end cap and is arranged in the internal space of the capsule that is in turn e.g. closed by said closure or peel-off seal.

Further, according to an embodiment, said end cap comprises a disinfectant (e.g. a compound, substance or material that is characterized by an antimicrobial property, i.e., inhibits and/or destroys microorganisms), wherein particularly said end cap is at least partially or completely formed out of said disinfectant, or comprises a coating comprising said disinfectant.

Further, as already indicated above, according to a preferred embodiment of the unit according to the present invention, the body is configured to be connected to a movable carrier (also denoted as carriage) of said apparatus in a releasable fashion, wherein particularly said carrier is movable with respect to a bottom of a housing of the apparatus so that the unit can particularly be moved together with said carrier inside said housing.

Further, according to a preferred embodiment of the unit according to the present invention, the body comprises a top side, wherein particularly said discontinuity of the lateral wall of the recess is arranged at the top side.

Further, according to a preferred embodiment of the unit according to the present invention, two through-holes are formed in the body of the unit on the top side of the body which are each configured to receive an associated pin protruding from said carrier, when the body is connected to the carrier, wherein particularly a face side of the respective pin is flush with the top side of the body when the unit is connected to the carrier.

Further, according to a preferred embodiment of the unit according to the present invention, the top side comprises a through-hole which indicates the position of a frangible inline seal of a first conduit of the connector.

Further, according to a preferred embodiment of the unit according to the present invention, for establishing said releasable connection between the body and the carrier, the body comprises two opposing internal surfaces (e.g. extending perpendicular to said top side), wherein said internal surfaces face each other, and wherein a latching means, particularly a latching nose, is provided on each internal surface for engaging with a complementary latching means, particularly a recess, of the carrier.

Further, according to a preferred embodiment of the unit according to the present invention, the body of the unit comprises a front side wall and an opposing back side wall. Particularly, the recess of the body for receiving the capsule is arranged on the front side wall.

Further, according to a preferred embodiment of the unit according to the present invention, the connector holder comprises a recess for receiving the connector (e.g. of the dialysis system, e.g. a Y-set in case of CAPD or a patient line, e.g. a single conduit, in case of APD), which recess is arranged on the top side and extends from the front side wall to the back side wall of the body of the unit.

Further, according to an embodiment of the unit, when the fastening lever of the unit is in the second position, it is configured to extend over the recess of the connector holder to secure the connector arranged in the recess of the connector holder, wherein particularly the fastening lever comprises a recess configured to receive the connector.

Further, according to a preferred embodiment of the unit according to the present invention, the recess of the connector holder comprises a Y-shape, namely an end region extending from the front side wall, which end region branches out into a first and a second region that extend from said end region to the back side wall, respectively, wherein said recess is configured to receive a (Y-shaped) connector comprising a first conduit and a second conduit, wherein the two conduits branch off from an end section of the connector, via which end section the connector is configured to be connected to said tubular fitting, wherein the end region of the recess is configured to receive said end section of the connector, and wherein the first region of the recess is configured to receive the first conduit, and wherein the second region of the recess is configured to receive the second conduit of the connector.

Particularly in case of APD, the connector comprises at least one conduit, particularly a single conduit, connected to the end section of the connector, wherein here said at least one conduit/single conduit is arranged in the second region of the recess while the first region stays empty.

Further, according to a preferred embodiment of the unit according to the present invention, the unit comprises a protrusion, particularly a U-shaped protrusion, protruding from the front side wall and extending along the end region of the recess of the connector holder on the front side wall, wherein said protrusion is configured to prevent an inclination of the end section of the connector with respect to an axial direction of the end section of the connector when said end section of the connector is inserted in said end region of the recess of the connector holder. Particularly, said protrusion comprises two opposing parallel sections that are integrally connected by a curved section which parallel sections and curved section are configured to butt against said end section of the connector in a form fitting manner to prevent said inclination.

Further, according to a preferred embodiment of the unit according to the present invention, the body of the unit comprises two holding means or holding parts arranged on the top side of the body, wherein the two holding means or parts are arranged on opposing sides of the end region of the recess for holding the end section of the connector in the end region of the recess when said end section is arranged in said end region of the recess.

Particularly, each of said two holding means or parts comprises a free end, wherein the respective free end protrudes past an associated side wall of the end region of the recess, which two side walls of said end region of the recess face each other.

Further, according to a preferred embodiment of the unit according to the present invention, the body comprises two holding means or two holding parts, wherein each of said holding means or parts forms a tooth structure, and wherein the respective holding means or part protrudes from an associated sidewall of the end region of the recess of the connector holder, wherein said two sidewalls face each other, and wherein said tooth structures are configured to prevent a movement of the end section of the connector in an axial direction when said end section of the connector is arranged in said end region of the recess of the connector holder and a force pulls said end section of the connector in said axial direction that particularly points away from said front side wall.

Further, according to a preferred embodiment of the unit according to the present invention, the body comprises a holding means or holding part arranged in the first region of the recess of the connector holder, which holding means or part is configured to clamp the first conduit of the connector when said first conduit is arranged in the first region of the recess of the connector holder, wherein particularly said holding means or part is configured to encompass said first conduit, and/or wherein the body comprises a holding means or a holding part arranged in the second region of the recess of the connector holder, which holding means or part is configured to clamp the second conduit of the connector (or said at least one conduit/single conduit) when said second conduit (or said at least one conduit/single conduit) is arranged in the second region of the recess of the connector holder, wherein particularly said holding means or part is configured to encompass said second conduit (or said at least one conduit/single conduit).

Further, according to a preferred embodiment of the unit according to the present invention, the body comprises a holding means or a holding part arranged in the first region of the recess of the conductor holder at the back side wall, which holding means or part is configured to clamp the first conduit of the connector when said first conduit is arranged in the first region of the recess of the connector holder, wherein particularly said holding means or part is configured to encompass said first conduit, wherein said holding means or part comprises a slot into which the first conduit can be pushed so that the first conduit is blocked for the passage of fluid through the first conduit, and/or wherein the body comprises a holding means or a holding part arranged in the second region of the recess of the connector holder at the back side wall, which holding means or part is configured to clamp the second conduit (or said at least one conduit/single conduit) of the connector when said second conduit (or said at least one conduit/single conduit) is arranged in the second region of the recess of the connector holder, wherein particularly said holding means or part is configured to encompass said second conduit (or said at least one conduit/single conduit), and wherein said holding means or part comprises a slot into which the second conduit (or the at least one conduit/single conduit) can be pushed so that the second conduit (or the at least one conduit/single conduit) is blocked for the passage of fluid through the respective conduit.

According to an alternative embodiment, the body comprises a holding means or holding part arranged in the first region of the recess of the connector holder at the back side wall, which holding means or part is configured to clamp the first conduit of the connector when said first conduit is arranged in the first region of the recess of the connector holder, wherein said holding means or part comprises a clamping surface and a pivotable clamping arm, wherein the clamping arm is configured to be pivoted towards the clamping surface into a clamping position so that the first conduit is blocked when it is clamped between said clamping surface and the clamping arm.

Likewise, according to an embodiment, the body can comprise a holding means or holding part arranged in the second region of the recess of the conductor holder at the back side wall, which holding means or part is configured to clamp the second conduit or said at least one conduit of the connector when said second conduit or said at least one conduit is arranged in the second region of the recess of the connector holder, wherein said holding means or part comprises a clamping surface and a pivotable clamping arm, wherein the clamping arm is configured to be pivoted towards the clamping surface into a clamping position so that the second conduit or said at least one conduit is blocked when it is clamped between said clamping surface and said clamping arm.

The clamping surfaces can be inclined with respect to the top side of the body.

Further, according to an embodiment, the clamping arm of the holding means or part of the first region of the recess of the conductor holder is configured to be pivoted towards the associated clamping surface by means of an actuating member of the apparatus. Further, the clamping arm of the holding means or part of the second region of the recess of the connector holder may also be configured to be pivoted towards the associated clamping surface by means of an actuating member of the apparatus.

Further, according to an embodiment, the holding means or part of the first region of the recess of the connector holder comprises a hook for engaging with the clamping arm so as to hold the clamping arm in its clamping position. Furthermore, according to an embodiment, the holding means or part of the second region of the recess of the connector holder comprises a hook for engaging with the clamping arm so as to hold the clamping arm in its clamping position.

Further, according to an embodiment, the unit comprises a release mechanism connected to or acting on the hook of the holding means arranged in the first region of the recess and/or connected to or acting on the hook of the holding means arranged in the second region of the recess, wherein the release mechanism is configured to displace the hook of the holding means arranged in the first region of the recess and/or the hook of the holding means arranged in the second region, particularly in the axial direction of the capsule, such that the clamping arm of the holding means arranged in the first region of the recess and/or the clamping arm of the holding means arranged in the second region of the recess is/are released from its/their clamping position, so that particularly the first conduit, the second conduit or the at least one conduit is unblocked, wherein particularly the release mechanism is a pressable button or comprises a pressable button.

Further, according to an embodiment, the holding means of the first region of the recess of the connector holder comprises a guiding arm for guiding said actuating member of the apparatus, and/or wherein the holding means of the second region of the recess of the connector holder comprises a guiding arm for guiding said actuating member of the apparatus.

Particularly in case the holding means described above comprises said pivotable clamping arms, it is also possible that the unit according to the present invention comprises a second receptacle that is rigidly or integrally connected to the body of the unit and cannot be removed from the body of the unit. In this regard, according to a further aspect of the present invention, the following unit is disclosed:

A unit for an apparatus for connecting and disconnecting a tubular fitting to a connector, comprising a body having a first receptacle and a second receptacle, wherein each of said receptacles is configured for holding an end cap of the tubular fitting, and wherein the body further comprises a connector holder configured to hold the connector. Here, particularly the second receptacle is rigidly and/or integrally connected to the body and cannot be removed from the body of the unit.

Further, particularly, this unit comprises an end cap arranged in said second receptacle formed by the body of the unit, wherein particularly said end cap is completely arranged in said second receptacle.

Further, particularly, said second receptacle is closed, particularly hermetically closed, by a closure comprising a peel-off seal, which peel-off seal is particularly attached to a circumferential face side of a lateral wall of the second receptacle, which lateral wall is formed by the body.

This unit with the second receptacle rigidly and/or integrally connected to the body may further comprise features disclosed in one of the claims 51 to 63 or 65 to 80.

Further, according to an embodiment of the unit according to the present invention, the body comprises a first and a second latching nose at the first region of the recess of the connector holder, wherein the respective latching nose is configured to engage with an associated actuating member of the apparatus so as to hold the respective actuating member in a pressed position, wherein particularly the first latching nose is configured to engage with a first actuating member of the apparatus that is configured to break said frangible inline seal of the first conduit. Further, particularly the first latching nose is configured to engage such with the first actuating member that the latter ensures the passage of fluid through the first conduit after breaking of the inline seal.

Further, particularly, the second latching nose is configured to engage with a second actuating member of the apparatus that is configured to press the first conduit in said slot of the first region of the recess.

Furthermore, according to an embodiment, the body comprises a third latching nose at the second region of the recess of the connector holder, wherein said third latching nose is configured to engage with an associated actuating member of the apparatus so as to hold said actuating member in a pressed position, wherein particularly the third latching nose is configured to engage with a third actuating member of the apparatus that is configured to press the second conduit in said associated slot of the second region of the recess.

Further, according to a preferred embodiment of the unit according to the present invention, the unit comprises a drip pan that protrudes from the front side wall of said body and is arranged below the end region of the recess as well as below the first receptacle to receive spilled fluid, wherein said drip pan comprises a plurality of dents for receiving spilled fluid.

Further, according to a preferred embodiment of the unit according to the present invention, the drip pan comprises a centering means in the form of a recess for receiving a guiding means of the apparatus, particularly in the form of a guiding pin. The drip pan may comprise a further recess for receiving said pin when the removable unit is arranged in the apparatus (with the removable unit being arranged in the first position).

Further, according to a preferred embodiment of the unit according to the present invention, the body of the unit comprises a Shore hardness in the range from 60 A to 100 A, particularly 70 A to 90 A, particularly 80 A.

Further, according to a preferred embodiment of the unit, the body comprises a material or is formed out of a material, which material is one of: polypropylene (PP); acrylonitrile butadiene styrene (ABS); a mixture of polycarbonate (PC) and acrylonitrile butadiene styrene (ABS).

Further, according to an embodiment of the unit, the capsule comprises a disinfectant (e.g. a compound, substance or material that is characterized by an antimicrobial property, i.e., inhibits and/or destroys microorganisms), wherein particularly the capsule is at least partially or completely formed out of said disinfectant. Further, according to an embodiment, the capsule comprises a coating comprising a disinfectant. Further, according to an embodiment, the closure comprises a disinfectant, wherein particularly the closure is at least partially or completely formed out of said disinfectant. Further, according to an embodiment, the closure comprises a coating comprising a disinfectant. Particularly, the coating can be arranged on an inside of the closure, which inside faces the internal space of the capsule. Further, according to an embodiment, an anti-bacterial agent or fluid is arranged in the internal space of the capsule.

Furthermore, according to an embodiment of unit, the unit comprises an ejection mechanism configured to eject the capsule out of the recess of the body unit.

According to yet another aspect of the present invention an apparatus configured to connect a tubular fitting to a connector and to disconnect the tubular fitting from the connector is disclosed, wherein the apparatus comprises a (e.g. removable) unit according to the present invention, and a holder assembly configured to hold or to accommodate insertion of the tubular fitting, wherein the unit is connected or connectable to a movable carrier of the apparatus, and wherein the carrier is movable with respect to the holder assembly.

Particularly, the (e.g. removable) unit according to the present invention is adapted to be used with such an apparatus for connecting and disconnecting a tubular fitting (e.g. of a catheter) to a connector (particularly to a fluid bag connector), which apparatus particularly uses an enclosed, movable carrier to which said unit is connected or connectable. Herein the invention is often described in terms of a fluid bag connector, but may also be used with any other connector. Furthermore, particularly, the notion catheter refers to medical catheters, but particularly also to all other structures or conduits via which a fluid can be delivered and which structures or conduits can be closed with a cap. Further, particularly, the notion cap or end cap refers to all kinds of closures that can be used in a reasonable manner in the framework of the present invention.

Particularly, said apparatus according to the present invention is designed to mechanically connect a Peritoneal Dialysis (PD) catheter to a dialysis bag during continuous ambulatory peritoneal dialysis (CAPD) procedure and also with APD, automated peritoneal dialysis when connected to a PD cycler. The apparatus is particularly intended for use by home dialysis patients, caregivers and health care professionals at home or within health care facilities.

Particularly, the unit ensures an antibacterial conduction of the connecting/disconnecing or fastening/unfastening (end caps) operations. It is preferably pre-filled/pre-assembled with the new (sterile) end cap that resides in the disposable capsule. Prior to the connecting operation, said body may be pre-filled with a sterile end cap, e.g. by inserting a capsule holding the end cap into the recess of the body of the unit, and is then assembled onto the moveable carrier of the apparatus. These components assist in connecting an e.g. PD catheter to an e.g. dialysis bag during a CAPD or APD procedure.

Further, according an embodiment of the apparatus, the movable carrier is movable (e.g. relative to said holder assembly) along a first direction (e.g. in transverse direction parallel to the holder assembly) from a first position to a second position, and from the second position to a third position, and particularly from the third position back to the first position.

Further, according an embodiment of the apparatus, the moveable carrier is mounted to a slider so that the carrier is movable along the first direction with respect to the slider, and wherein the slider is movable (back and forth) along a second direction which runs perpendicular to the first direction.

Further, according an embodiment of the apparatus, when the carrier is in the first position, the carrier is movable away from the holder assembly by moving the slider away from the holder assembly along the second direction so as to unfasten an end cap from the tubular fitting, when the end cap is received in the first receptacle (or engaged with the first receptacle) and fastened to the tubular fitting and when said tubular fitting is inserted into the holder assembly. Further, according to an embodiment of the apparatus, when the movable carrier is in the second position with respect to the slider, the carrier is movable towards the holder assembly by moving the slider towards the holder assembly along the second direction to connect a connector inserted into the connector holder to the tubular fitting (so that particularly a flow connection can be established between the tubular fitting and the connector). Further, according to an embodiment of the apparatus, when the movable carrier is in the second position with respect to the slider, the carrier is movable away from the holder assembly by moving the slider away from the holder assembly along the second direction so as to disconnect the connector from the tubular fitting. Further, according to an embodiment, when the movable carrier is in the third position with respect to the slider, the movable carrier is movable towards the holder assembly by moving the slider towards the holder assembly along the second direction to fasten the end cap arranged in the internal space of the capsule (with the closure removed from the capsule) to the tubular fitting.

Further, according an embodiment of the apparatus, the apparatus can comprise a locking mechanism configured to prevent movement of the slider when a protective cap (not shown) closing the connector initially has not been connected to a movable locking bar of the locking mechanism. For this, the protective cap can comprise a loop that can be put over the locking bar.

Particularly, the locking mechanism can be released to allow moving the slider, when the locking bar is moved from a first position of the locking bar to a second position of the locking bar. Particularly, the locking bar is pretensioned towards the first position and is configured to be held in the second position by laying said loop of the protective cap around the locking bar. In this way, when the movable carrier is moved away from the holder assembly in the first position of the movable carrier, the protective cap can be pulled off the connector by means of the locking bar.

Further, according to an embodiment of the apparatus, the apparatus comprises a bottom. Further, according to an embodiment, the apparatus comprises a housing that comprises said bottom of the apparatus. The housing can further comprise a cover that is connected or connectable to the bottom (e.g. so that the cover is pivotable with respect to the bottom of the housing).

Furthermore, in an embodiment of the apparatus, the cover is configured to be opened and/or removed from the housing (e.g. from the bottom of the housing). Furthermore, according an embodiment of the apparatus, the cover is at least partially or completely transparent, particularly so as to be able to observe said fastening/unfastening of the end caps and said connecting of the tubular fitting to the connector or said disconnecting of the tubular fitting from the connector.

Particularly, according to an embodiment of the apparatus, the housing is configured to receive and particularly enclose the unit that is configured to hold the connector and end caps to be fastened to or unfastened from the tubular fitting.

Particularly, according to an embodiment of the apparatus, said holder assembly comprises a body having an opening configured to receive the tubular fitting.

Further, according to an embodiment, the holder assembly or body of the holder assembly is fixed to the bottom or housing of the apparatus (e.g. integrally or by fastening elements), and particularly cannot move with respect to the bottom or housing. When the tubular fitting is received in the holder assembly (particularly in said opening of the body of the holder assembly) as intended, said member for opening/closing the tubular member protrudes out of the housing and is accessible for the user so that the user can open/close the tubular fitting for the passage of fluid by means said member.

Particularly, in an embodiment, the cover is connected to a lateral wall of the housing, wherein said lateral wall of the housing is connected to the bottom of the housing. The cover of the housing can be mounted to the lateral wall (or to the bottom of the housing) such that it is pivotable to open or close the housing. When the housing is closed, the cover covers the unit and particularly the capsule. When the housing is open, the unit and/or capsule can be removed from the apparatus (particularly from the movable carrier).

Further, according an embodiment of the apparatus, the slider is movable (e.g. along the second direction) with respect to the bottom of the apparatus.

Particularly, according to an embodiment of the apparatus, the slider is mounted to the bottom of the apparatus such that it is movable with respect to the bottom back and forth along the second direction.

According to an embodiment of the apparatus the bottom comprises a first (e.g. upper) bottom wall and a second (e.g. lower) bottom wall, wherein the bottom of the apparatus comprises an interspace arranged between the first bottom wall and the second bottom wall, and wherein the first bottom wall comprises a top side facing away from the second bottom wall (or from the interspace). Thus, in other words, the bottom of the apparatus can be a double bottom.

Furthermore, according to an embodiment of the apparatus, the slider comprises a first portion arranged on the top side of the first bottom wall. Further, particularly, the slider comprises a second portion connected to the first portion of the slider. Furthermore, according to an embodiment of the apparatus, the movable carrier is mounted to the first portion of the slider. Further, according to an embodiment of the apparatus, the second portion of the slider extends through a slot formed in the first (e.g. upper) bottom wall into the interspace of the bottom of the housing of the apparatus. Further, according to an embodiment of the apparatus, the second portion of the slider (and therewith the slider) is guided by a guide rail that is arranged in the interspace and/or that is connected to the second (e.g. lower) bottom wall of the bottom of the apparatus. Particularly, in an embodiment of the apparatus, the second portion of the slider can comprise a first part connected (e.g. integrally) to the first portion of the slider. Further, in an embodiment, the second portion of the slider can comprise a separate second part that is connected to the first part (e.g. by screws) and that is guided by the guide rail.

Further, according an embodiment of the apparatus, the apparatus comprises an actuating element configured to move the moveable carrier and/or the slider.

Furthermore, according to an embodiment of the apparatus, the actuating element is formed as an elongated lever having a first end section and an opposing second end section. Particularly, the first end section of the actuating element is arranged in the interspace of the bottom of the apparatus and is pivotably mounted to the bottom of the apparatus particularly to the second bottom wall and/or to the first bottom wall.

Furthermore, according to an embodiment of the apparatus, the second end section of the actuating element forms a handle for pivoting the actuating element manually. Particularly, the second end section or handle protrudes out of the bottom and/or housing of the apparatus.

Furthermore, according to an embodiment, the actuating element comprises a connecting section that connects the first end section of the actuating element to the second end section of the actuating element. Particularly, the connecting section is coupled to the second portion of the slider such that the slider is movable (e.g. back and forth) along the second direction by pivoting the actuating element. According to an embodiment, for coupling the actuating element to the second portion of the slider, the actuating element comprises an oblong hole in the connecting section, wherein the second portion of the slider can comprise an element (e.g. formed by said first part of the second portion of the slider or by a portion of said first part) that engages with the oblong hole and thus moves the slider along the second direction when the actuating element is pivoted.

Particularly, according to an embodiment, the actuating element is configured to be pivoted back and forth between a first position and a second position of the actuating element, wherein when the actuating element is pivoted (e.g. from the second position) towards the first position, the slider moves towards the holder assembly along the second direction and when the actuating element is pivoted (e.g. from the first position) towards the second position, the slider moves away from the holder assembly along the second direction.

Furthermore, according to an embodiment, the actuating element comprises two latching arms protruding from the connecting section of the actuating element. Further, particularly, the apparatus comprises a member arranged in the interspace (said member can be connected to the first and/or second bottom wall) that forms a stop for the actuating element (e.g. when the actuating element resides in the first position), and wherein particularly said member is configured to engage with the latching arms (e.g. to arrest the actuating element) when the actuating element is arranged in the first position of the actuating element.

Further, according an embodiment of the apparatus, the apparatus comprises a hook element that is particularly connected to the bottom of the apparatus (e.g. to the first or upper bottom wall of the bottom on the top side of the first bottom wall) and that is configured to engage with the through-opening of the closure for removing the closure from the capsule when the carrier is in the second position and/or moved away from the holder assembly along the second direction. Further, according to an embodiment of the apparatus, the apparatus comprises a further hook element that is particularly connected to the bottom of the apparatus (e.g. to the first or upper bottom wall of the bottom on the top side of the first bottom wall) and that is configured to engage with the further through-opening of the closure for removing the closure from the capsule when the carrier is in the second position and/or moved away from the holder assembly along the second direction

Further, according an embodiment of the apparatus, the apparatus comprises a spring (e.g. an unwindable/rewindable spring) for moving the carrier along the first direction with respect to the slider from the first position to the second position and from the second position to the third position. Particularly, in an embodiment, the movable carrier is configured to be moved from the third position to the first position with respect to the slider manually (using e.g. the movable carrier member as an actuating element/handle) to tension the spring.

Further, according an embodiment of the apparatus, the apparatus comprises a pivotable stopper mounted to the slider (particularly to the first portion of the slider), wherein the stopper is configured to stop the carrier in the first and in the second position, wherein particularly the stopper is configured to be pivoted to release the carrier when the carrier is in the first or in the second position, and wherein the stopper is pivotable by moving the slider away from the holder assembly along the second direction (e.g. by means of the pivotably mounted actuating element) such that the stopper engages with a pin that protrudes from the bottom of the apparatus (particularly from the top side of the first bottom wall) whereby the stopper is pivoted and thereby releases the carrier. Particularly, once the carrier is released by the pivotable stopper (in the first and in the second position) it moves under the action of the spring along the first direction with respect to the slider. Particularly, according to an embodiment, the spring and the pivotable stopper are covered by the movable carrier which is arranged on the slider (e.g. on the first portion of the slider).

Furthermore, according to an embodiment of the apparatus, the stopper is connected via a spring element to the slider (particularly to the first portion of the slider), wherein the spring element is configured to exert a restoring force on the stopper for pivoting the stopper back into a stopping position in which the stopper stops the carrier (e.g. in the first and second position of the movable carrier).

Furthermore, according to an embodiment of the apparatus, for engaging with the pin, the pivotable stopper comprises an end portion that extends through a curved slot formed in the slider. Particularly said curved slot is formed in the first portion of the slider. Particularly, the end portion of the stopper is configured to contact the pin when the slider is moved along the second direction away from the holder assembly so that the stopper is pivoted and thereby releases the carrier (see above).

Further, according an embodiment of the apparatus, the apparatus comprises a tubular fitting that is particularly configured to be received by the holder assembly, particularly by an opening of the body of the holder assembly of said apparatus. Thus, particularly, the tubular fitting is configured to be inserted into said opening of the body of the holder assembly.

Further, according to an embodiment, the tubular fitting comprises a member which is configured to be actuated so as to open or close the tubular fitting (or catheter) for allowing or preventing the passage of fluid through the tubular fitting.

Further, according to an embodiment, said member of the tubular fitting is a rotatable member that is configured to be actuated by rotating it (e.g. about an axis along which the tubular fitting or catheter extends), wherein upon rotation of the member of the tubular fitting, the tubular fitting is either closed so that no fluid can pass through the tubular fitting or opened so that fluid can pass through the tubular fitting depending on the direction of said rotation. Other ways of allowing/preventing passage of fluid through the tubular fitting are also conceivable.

Further, according to an embodiment of the apparatus, the tubular fitting is configured such that an end cap can be fastened to the tubular fitting by plugging the end cap into said opening, and wherein the tubular fitting is configured such that an end cap can be unfastened from tubular fitting by pulling the end cap out of said opening.

Further, according to an embodiment of the apparatus, the tubular fitting is configured such that a connector can be connected to the tubular fitting by plugging the connector into said opening of the tubular fitting, and wherein the tubular fitting is configured such that a connector can be disconnected from the tubular fitting by pulling the connector out of said opening of the tubular fitting.

Further, preferably, said plugging and/or pulling is irrotational.

Further, according to an embodiment of the tubular fitting according to the invention, the tubular fitting is formed as an adapter, which comprises a first recess at a first end of the adapter, which first recess comprises an internal thread configured to be rotationally fastened to an external thread of a first portion of a catheter, and wherein the adapter comprises a shroud at an opposite second end of the adapter, which shroud surrounds a second recess of the adapter into which a protrusion of the adapter protrudes, which protrusion comprises said opening of the adapter such that the shroud surrounds said protrusion and said opening of the adapter, wherein the shroud is coaxially arranged with respect to said protrusion.

To describe the adapter function in more detail, the tubular fitting will be described below in terms of a first and a second portion of the tubular fitting.

Further, according an embodiment of the apparatus, the tubular fitting comprises a first portion. Particularly, the first portion is configured to be connected to a catheter or may form part of such a catheter, e.g. of a transfer-set (e.g. a mini-set). Furthermore, the tubular fitting comprises an adjacent second portion comprising said opening of the tubular fitting for connecting with a connector or for fastening an end cap to the second portion/tubular fitting.

Further, according to an embodiment of the apparatus, the second portion is configured such that an end cap can be fastened to the second portion by plugging the end cap into said opening of said second portion (e.g. upon said movement of the cradle assembly or removable unit towards the holder assembly when the cradle assembly/removable unit is in the third position), and wherein the second portion is configured such that an end cap can be unfastened from the second portion by pulling the end cap out of said opening, (e.g. upon said movement of the cradle assembly or removable unit away from the holder assembly when the cradle assembly/removable unit is in the first position).

Further, according to an embodiment of the apparatus, the second portion is configured such that a connector can be connected to the second portion by plugging the connector into said opening of said second portion (e.g. upon said movement of the movable carrier or unit towards the holder assembly when the movable carrier/unit is in the second position), and wherein the second portion is configured such that a connector can be disconnected from the second portion by pulling the connector out of said opening (e.g. upon said movement of the movable carrier/unit away from the holder assembly when the movable carrier/unit is in the second position).

Further, particularly, as already indicated, said plugging and/or pulling is preferably irrotational (i.e. does not involve any rotation of the second portion/tubular fitting or of the respective end cap).

Further, according to an embodiment of the apparatus, the tubular fitting comprises a removable clamp for arresting the connector with respect to the tubular fitting (e.g. with respect to the second portion) when the connector is connected to the tubular fitting (e.g. to the second portion), wherein particularly the clamp is configured to be connected to the tubular fitting (e.g. to the second portion), particularly by means of a clip-on connection, wherein particularly the clamp encompasses the tubular fitting (e.g. the second portion) when it is connected to the tubular fitting (e.g. to the second portion).

Further, according to an embodiment of the apparatus, the second portion is formed as an adapter (see also above), which comprises a first recess at a first end of the adapter, which first recess comprises an internal thread configured to be rotationally fastened to an external thread of the first portion, particularly such that a lumen surrounded by the first portion is in flow communication with a lumen surrounded by the adapter. Preferably, the first recess comprises a conical shape. Further, preferably, the external thread is formed on a conical section of the first portion. Furthermore, preferably, the adapter comprises a shroud, preferably a bell-shaped shroud, at an opposite second end of the adapter, which shroud surrounds a second recess of the adapter into which second recess a protrusion of the adapter protrudes, which protrusion preferably comprises said opening of the adapter such that the shroud surrounds said protrusion and said opening of the adapter. Further, the shroud is preferably coaxially arranged with respect to said protrusion.

Further, according to an alternative embodiment of the apparatus, the second portion may be integrally connected to the first portion (which in turn may be connected to said catheter), particularly such that the two portions (i.e. their lumina) are in flow communication with each other.

Here, particularly, the second portion comprises a shroud, which shroud surrounds a recess of the second portion into which a protrusion of the second portion protrudes, which protrusion comprises said opening of the second portion such that the shroud surrounds said protrusion and said opening of the second portion, wherein the shroud is coaxially arranged with respect to said protrusion.

Further, according to an embodiment of the apparatus, the clamp is configured to cover a portion of the shroud and of the second recess when the clamp is connected to the tubular fitting (e.g. to the second portion), wherein the clamp is configured to engage behind a portion of the connector when the connector is connected to the tubular fitting (e.g. to the second portion) so that the connector cannot be disconnected from the tubular fitting (e.g. from the second portion).

Further, according to an embodiment of the apparatus, the clamp is configured to be connected to the tubular fitting (e.g. to the second portion) and/or removed from the tubular fitting (e.g. from the second portion) when the tubular fitting is arranged in the holder assembly of the apparatus.

Further, according to an embodiment of the apparatus, the clamp comprises two arms extending from a base of the clamp for encompassing the tubular fitting, particularly its second portion.

Further, according to an embodiment of the apparatus, the clamp comprises an inner protrusion (e.g. at a position where the shroud ends) for insertion into a circumferential groove of the tubular fitting, particularly of the second portion, when the clamp is connected to the tubular fitting, e.g. to the second portion.

Further, according to an embodiment of the apparatus, the clamp comprises a flap for removing the clamp from the tubular fitting, which flap is connected to the base, particularly via a hinge, wherein particularly, for forming said hinge, the clamp comprises a through-hole arranged between the base and the flap.

Particularly, according to yet another aspect of the present invention, a catheter system is disclosed which comprises a catheter and a tubular fitting and/or a connector as described herein, wherein particularly the first portion of the tubular fitting described below is configured to be in fluid communication with a lumen of the catheter (or can be brought in fluid communication with said lumen of the catheter, particularly by means of said member described herein).

Further, according to an embodiment of the apparatus, the apparatus comprises a removable clamp configured to arrest the connector with respect to the tubular fitting when the connector is connected to the tubular fitting. Such a removable clamp is particularly useful for an APD when the tubular fitting and the connector are connected and removed from the apparatus. This ensures that the connection between these two components can be maintained.

Particularly, the removable clamp is configured to be connected to the tubular fitting, particularly by means of a clip-on connection, wherein particularly the clamp encompasses the tubular fitting when it is connected to the tubular fitting.

Further, according to an embodiment of the apparatus, the clamp is configured to cover a portion of a shroud and of an opening of the tubular fitting when the clamp is connected to the tubular fitting, wherein the clamp is configured to engage behind a portion of the connector when the connector is connected to the tubular fitting so that the connector cannot be disconnected from the tubular fitting.

Further, according to an embodiment of the apparatus, the clamp is configured to be connected to the tubular fitting and/or removed from the tubular fitting when the tubular fitting is arranged in a holder assembly of an apparatus.

Further, according to an embodiment of the apparatus, the removable clamp comprises two arms extending from a base of the clamp for encompassing the tubular fitting (e.g. the second portion of the tubular fitting).

Further, according to an embodiment of the apparatus, the clamp comprises a protrusion for insertion into a circumferential groove of the tubular fitting when the clamp is connected to the tubular fitting.

Further, according to an embodiment of the apparatus, the clamp comprises a flap for removing the removable clamp from the tubular fitting, which flap is connected to the base, particularly via a hinge, wherein particularly, for forming said hinge, the clamp comprises a through-hole arranged between the base and the flap.

Further, particularly, the adapter/tubular fitting according to the invention is designed to transform a rotational Luer movement into a linear movement so that connecting/disconnecting or fastening/unfastening can be performed in a rotation free manner by means of merely linear plugging or pulling movement.

Particularly, the adapter/tubular fitting according to the invention is intended to mate two devices together and is—according to an embodiment—either protected by the closure cap (also denoted end cap) or connected to the dialysis bag (or any tubing connected to a drug or a drug delivery system) during the therapy. Particularly, the adapter is fastened to the catheter (or e.g. a transfer set, for instance mini-set) of the patient and particularly rests there for the e.g. 6 months said catheter (or e.g. a transfer- or mini-set) is in use.

Further, particularly, the end cap according to the present invention is intended to be used for closure of the adapter when no dialysis bag is attached to perform the therapy. Particularly, it is held in place with a secured sealed locking mechanism.

Particularly, the apparatus comprising the above described components is intended to be used as a patient aid in home dialysis therapy. Particularly, it supports the connection of the e.g. catheter to the e.g. dialysis bag. It performs this connection mechanically in an easy manner, therefore providing a convenient alternative to the conventional connection performed completely manually.

Further, according to an embodiment of the apparatus according to the invention, said body of the unit can be (e.g. manually) released from a movable carrier of the cradle assembly that is moveable in said positions. Further, particularly, the connected catheter-dialysis fluid line (comprising in an embodiment at least the tubular fitting and the connector as well as particularly conduits connected thereto) can be removed from the housing when the movable carrier is in the second position and has particularly been moved towards the holder assembly, e.g. in order to allow application of the device for APD.

Further, optionally, the holder assembly and/or the tubular fitting may be configured to move together with the slider when the movable carrier/unit is in the second position such that a member of a tubular fitting, which tubular fitting is inserted into the holder assembly, protrudes at least partially out of the housing (e.g. through a through hole of the housing) when the tubular fitting is connected to a connector that is inserted into the body of the unit and such that said member is arranged inside the housing (e.g. in a compartment surrounded by the housing) when said tubular fitting is disconnected from said connector, wherein said member is configured to be actuated so as to open or close the tubular fitting (or catheter) for allowing or preventing the passage of fluid through the tubular fitting. Particularly, when the holder assembly is non-moving, the tubular fitting may also be configured to slide with respect to the holder assembly, so that said member can be retracted into the housing or pushed out of the housing.

Further, optionally, the apparatus may be configured to prevent retracting of the tubular fitting for disconnecting the latter from the connector (e.g. by preventing retracting of the holder assembly into the housing) when the tubular fitting clamp (e.g. said member of the tubular fitting described herein) is not closed.

Hence, to ensure safety, the tubular fitting/catheter may be configured such that it cannot be opened for allowing passage of fluid through the fitting/catheter from outside the housing when the tubular fitting is not connected to the connector and is not fastened to an end cap.

Further, optionally, the holder assembly may also be configured to move together with the slider when the movable carrier is in the third position such that a member of a tubular fitting, which tubular fitting is inserted into the holder assembly, protrudes—at least partially—out of the housing (e.g. through said through hole of the housing) when the tubular fitting is fastened to an end cap that is received in the opened second receptacle, wherein said member can be actuated so as to close the tubular fitting (or catheter) for preventing passage of fluid through the tubular fitting.

Further, according to an embodiment, the apparatus comprises an actuating member, e.g. a first actuating member (e.g. as described above), that is configured to be manually actuated to break a frangible inline seal (e.g. of the first conduit of the connector) when the movable carrier or the unit is in the second position and a connector inserted into the connector holder is connected to a tubular fitting inserted into the holder assembly. Preferably, the first actuating member comprises a pushable button arranged on the housing (particularly on the cover), wherein upon pushing said button, the first actuating member moves downwards and breaks said frangible inline seal of the first conduit arranged in the first region of the recess of the connector holder.

Further, particularly, the apparatus comprises an actuating member, e.g. a second actuating member (e.g. as described above), that is configured to be manually actuated to interrupt the passage of fluid through the first conduit of the connector arranged in the first region of the recess of the connector holder when the moveable carrier (or unit) is in the second position and the connector inserted into the recess of the connector holder is connected to a tubular fitting inserted into the holder assembly. Preferably, the second actuating member comprises a pushable button arranged on the housing (particularly on the cover), wherein upon pushing said button, the second actuating member moves downwards and pushes the first conduit into said slot so that the first conduit is interrupted. Alternatively, the second actuating member is configured to pivot the clamping arm of the holding means arranged in the first region of the recess of the connector holder.

Further, particularly, the apparatus comprises a third actuating member (e.g. as described above) that is configured to be manually actuated to interrupt the passage of fluid through the second conduit of the connector being arranged in the second region of the recess when the movable carrier or the unit is in the second position and the connector inserted into the recess of the connector holder is connected to a tubular fitting inserted into the holder assembly.

Further, the third actuating member preferably comprises a pushable button arranged on the housing (particularly on the cover), wherein upon pushing said button, the third actuating member moves downwards and pushes the second conduit into the associated slot so that the second conduit is interrupted. Alternatively, the third actuating member is configured to pivot the clamping arm of the holding means arranged in the second region of the recess of the connector holder.

Preferably, the first conduit is connected to a fluid bag and the second conduit to a fluid waste bag.

Further, particularly, due to said latching noses, the buttons and/or actuating members are configured to remain in a different (e.g. lower position) after they have been pushed by a user, e.g. in order to indicate to the patient/user that they have already been operated.

Particularly, all the three buttons stay in a half-down or down position when pushed during the therapy in order to indicate to the patient that they have already been operated.

Further, particularly, the apparatus comprises a guiding means (e.g. guide rail) for guiding said movement of the slider towards and away from the holder assembly as described above. Further, particularly, said guiding means (e.g. guide rail) may also be configured for guiding said movement of the holder assembly.

Further, particularly, the apparatus can also comprise one or several actuators to move the movable carrier and/or the slider (and particularly also the holder assembly). Furthermore, the apparatus may comprise an electronic control unit for controlling said actuator(s). Said electronic control unit is preferably programmable in an embodiment of the apparatus so that the movement of the carrier and/or slider (and particularly also of the holder assembly) is programmable.

Further, particularly, the apparatus may comprise an antibacterial radiation source (e.g. an ultraviolet light source) for reducing contamination with germs.

Further, particularly, the apparatus may comprise a surface comprising a disinfectant admixture, particularly comprised by a coating of said surface or an additive to a molding material of said surface in order to reduce contamination with germs.

Furthermore, according to an embodiment of the apparatus, the apparatus can comprise an ejection mechanism configured to eject the capsule out of the recess of the body of the unit.

Yet a further aspect of the present invention relates to a capsule, particularly a disposable capsule, particularly for use with a unit or an apparatus according to the present invention. The capsule can also be used with an apparatus according to the present invention that comprises a unit that cannot be removed from the apparatus.

Particularly, the capsule is configured to used with a unit of an apparatus, which apparatus is configured to connect a tubular fitting to a connector and to disconnect the tubular fitting from the connector.

Particularly, the capsule comprises an internal space and an end cap arranged in said internal space.

Further, particularly, the capsule is configured to be connected to said unit, particularly to a body of the unit, in a releasable fashion.

Particularly, the capsule can comprise any of the features already described above.

Furthermore, the end cap that is arranged in the internal space of the capsule can comprise any of the features of the end cap already described above.

Furthermore, according to an embodiment of the capsule, the capsule comprises a lateral wall surrounding said internal space of the capsule in a peripheral direction of the capsule, wherein particularly the lateral wall can be cylindrical or can comprise a cylindrical portion. Further, particularly, the lateral wall of the capsule comprises an outside facing away from the internal space of the capsule, and particularly an inside facing the end cap residing in the internal space of the capsule. Particularly, the outside of the capsule can form a cylindrical surface of the capsule.

Furthermore, according to an embodiment of the capsule, the capsule comprises a bottom wall connected to the lateral wall of the capsule, wherein the lateral wall of the capsule extends from the bottom wall of the capsule in an axial direction of the capsule towards a face side of the lateral wall of the capsule, wherein the face side faces away from an outside of the bottom wall of the capsule. Particularly, the bottom wall of the capsule and/or its outside can be circular.

Furthermore, according to an embodiment of the capsule, the capsule comprises an axial protrusion protruding in the axial direction of the capsule from the bottom wall on the outside of the bottom wall of the capsule, wherein particularly the axial protrusion is configured to engage with a portion of a recess of the body, particularly in a form fitting manner.

Furthermore, according to an embodiment of the capsule, the axial protrusion is configured for self-positioning of the capsule with respect to the body of the unit. Particularly, the axial protrusion is configured to engage with said portion of the recess of the body so that a self-positioning of the capsule (e.g. with respect to the peripheral direction along which the lateral wall of the capsule extends) occurs upon engagement of the axial protrusion with said portion of the recess of the body.

Furthermore, according to an embodiment of the capsule, the axial protrusion tapers, particularly in a direction perpendicular to the axial direction of the capsule.

Furthermore, according to an embodiment of the capsule, the axial protrusion is wedge-shaped, particularly tapering in a direction perpendicular to the axial direction of the capsule.

Furthermore, according to an embodiment of the capsule, the axial protrusion of the capsule comprises a planar first surface and a planar second surface, the first surface facing away from the second surface, wherein particularly the first and the second surface extend perpendicular to the outside of the bottom wall of the capsule.

Furthermore, according to an embodiment of the capsule, the axial protrusion comprises a cylindrical shape (or at least a cylindrical portion).

Furthermore, according to an embodiment of the capsule, the end cap comprises a protrusion having a basis and a head, wherein particularly the head forms a free end of the protrusion of the end cap, and wherein particularly the head comprises a diameter perpendicular to a direction in which the protrusion protrudes from the end cap, wherein this diameter is larger than a diameter of the basis perpendicular to said direction.

Further, according to an embodiment of the capsule, the head is configured to engage behind an edge of a first receptacle of the unit, so that the tubular fitting can be pulled off the end cap when the end cap has its head engaged with said edge of the first receptacle.

Furthermore, according to an embodiment of the capsule, said axial protrusion of the capsule surrounds a portion of the internal space of the capsule, wherein said protrusion of the end cap is arranged in said portion of the internal space of the capsule so that particularly the end cap residing in the internal space of the capsule comprises a fixed position.

Furthermore, according to an embodiment of the capsule, the capsule comprises a lateral protrusion protruding from the lateral wall of the capsule (particularly in a radial direction of the capsule) on an outside of the lateral wall of the capsule, wherein the lateral protrusion particularly extends in the peripheral direction of the capsule.

Furthermore, according to an embodiment of the capsule, the lateral protrusion is spaced apart from the face side of the lateral wall of the capsule in the axial direction of the capsule.

Furthermore, according to an embodiment of the capsule, the lateral protrusion is spaced apart from the outside of the bottom wall of the capsule in the axial direction of the capsule.

Furthermore, according to an embodiment of the capsule, the lateral protrusion is arranged adjacent the bottom wall of the capsule (particularly adjacent the outside of the bottom wall).

Furthermore, according to an embodiment of the capsule, the lateral protrusion is arranged adjacent the face side of the lateral wall.

Furthermore, according to an embodiment of the capsule, the lateral protrusion is a circumferential (e.g. annular) lateral protrusion.

Furthermore, according to an embodiment of the capsule, the lateral protrusion comprises at least one gap.

Furthermore, according to an embodiment of the capsule, the lateral protrusion comprises a first gap and a second gap.

Furthermore, according to an embodiment of the capsule, the lateral protrusion comprises a third gap, wherein particularly the third gap branches off the first gap in the peripheral direction of the capsule or is adjacent to the first gap.

Furthermore, according to an embodiment of the capsule, the lateral protrusion comprises a first radial projection adjacent to the first gap.

Furthermore, according to an embodiment of the capsule, the lateral protrusion comprises a second radial projection adjacent to the first gap, wherein particularly the second radial projection is arranged adjacent the third gap, wherein particularly the second radial projection is shaped as an arrow pointing essentially in a radial direction of the capsule.

Furthermore, according to an embodiment of the capsule, the capsule comprises a further lateral protrusion protruding from the lateral wall of the capsule on the outside of the lateral wall, wherein the further lateral protrusion extends in the peripheral direction of the capsule.

Furthermore, according to an embodiment of the capsule, the further lateral protrusion is spaced apart from said lateral protrusion in the axial direction of the capsule, wherein particularly said further lateral protrusion faces said lateral protrusion in the axial direction of the capsule.

Furthermore, according to an embodiment of the capsule, the further lateral protrusion is arranged adjacent the face side of the lateral wall of the capsule. Particularly, the further lateral protrusion can be flush with the face side of lateral wall of the capsule.

Furthermore, particularly, the face side of the lateral wall of the capsule delimits an opening of the capsule via which the internal space of the capsule is accessible, wherein said opening can be closed by means of a closure, see e.g. below).

Further, particularly, the respective radial direction of the capsule runs perpendicular to the axial direction of the capsule.

Furthermore, according to an embodiment of the capsule, the further lateral protrusion is a circumferential (e.g. annular) lateral protrusion.

Furthermore, according to an embodiment of the capsule, the lateral protrusion is configured to engage with a groove formed by the recess of the body of the unit, particularly for fixing the capsule with respect to the body when the capsule is arranged in the recess of the body.

Furthermore, according to an embodiment of the capsule, said internal space of the capsule is closed, particularly hermetically closed, by a closure attached to the lateral wall, wherein the closure is particularly attached to the face side (and particularly to the further lateral protrusion) of the lateral wall of the capsule.

Furthermore, according to an embodiment of the capsule, the closure is or comprises a peel-off seal. Furthermore, according to an embodiment of the capsule, the peel-off seal is attached to the face side of the lateral wall of the capsule.

Furthermore, according to an embodiment of the capsule, the closure comprises a through-opening, wherein particularly the through-opening can be one of: an oblong hole, a quadrangular hole, a square hole, a circular hole

Particularly, according to an embodiment, the through-opening is formed in an (e.g. flexible) edge region of the closure, wherein particularly said edge region is a free edge region, and wherein particularly the edge region protrudes from the lateral wall of the capsule perpendicular to the axial direction of the capsule.

Furthermore, according to an embodiment of the capsule, the closure comprises a further through-opening, wherein particularly the further through-opening can be one of: an oblong hole, a quadrangular hole, a square hole, a circular hole, wherein particularly the further through-opening is formed in said edge region of the closure.

Furthermore, according to an embodiment of the capsule, said edge region comprises a section of an edge of the closure, which section extends linearly, wherein the through-opening is arranged adjacent said section of the edge of the closure, and wherein particularly said further through-opening is arranged adjacent said section of the edge.

Particularly, according to an embodiment of the capsule, the two through-openings are spaced apart from each other in a direction extending parallel to said section of the edge of the closure.

Furthermore, according to an embodiment of the capsule, the two through-openings are equidistantly spaced apart from said section of the edge in a direction perpendicular to said section of the edge.

Furthermore, according to an embodiment of the capsule, the through-opening is configured to receive a hook element, particularly a hook element of an apparatus to which the unit is connectable or connected for removing, particularly peeling off, the closure (e.g. peel-off seal). Furthermore, according to an embodiment, the further through-opening is configured to receive a further hook element of said apparatus for removing the closure, particularly for peeling off the closure (e.g. peel-off seal).

Furthermore, according to an embodiment of the capsule, the closure comprises a first slot adjacent to the through-opening and a second slot adjacent to the further through-opening, wherein the through-opening and the further through-opening are arranged between the first slot and the second slot in the peripheral direction, and wherein particularly the first slot and the second slot extend perpendicular to the section of the edge of the closure.

Furthermore, according to an embodiment of the capsule, said closure comprises a flexible strip connected to the peel-off seal, which strip comprises a free end section that forms a handle by means of which the peel-off seal can be peeled off the lateral wall of the capsule, particularly from outside or inside the housing of the apparatus to which the unit is connectable or connected, wherein particularly said free end section is configured to be arranged between a bottom and a cover of said housing such that the free end section of the strip protrudes out of the housing.

Furthermore, according to an embodiment of the capsule, the capsule comprises an indicator that is configured to indicate whether the capsule is unused.

Furthermore, according to an embodiment of the capsule, the lateral wall of the capsule comprises at least one latching nose or at least one latching recess.

Particularly, in an embodiment of the capsule according to the present invention, said end cap is completely arranged in said internal space of the capsule. Particularly, the capsule encloses the end cap (e.g. hermetically).

Furthermore, according to an embodiment of the capsule, the end cap is configured to be connected to a tubular fitting (e.g. as described herein) so as to close the tubular fitting.

Furthermore, according to an embodiment of the capsule, the end cap comprises a flexible carrier member or carrier means, particularly a sponge, that carries an anti-bacterial agent or fluid.

Furthermore, according to an embodiment of the capsule, the end cap comprises a disinfectant, wherein particularly said end cap is at least partially or completely formed out of said disinfectant, or comprises a coating comprising said disinfectant.

Furthermore, according to an embodiment of the capsule, the capsule comprises a disinfectant, wherein particularly said capsule is at least partially or completely formed out of said disinfectant, or comprises a coating comprising said disinfectant.

Further, according to an embodiment, the closure comprises a disinfectant, wherein particularly said closure is at least partially or completely formed out of said disinfectant, or comprises a coating comprising said disinfectant.

Furthermore, according to an embodiment, the coating can be arranged on an inside of the closure, which inside faces the internal space of the capsule. Furthermore, according to an embodiment an anti-bacterial agent or fluid is arranged in the internal space of the capsule.

Furthermore, according to an embodiment of the capsule, the end cap is formed out of a thermoplastic elastomer or comprises a thermoplastic elastomer, wherein particularly said thermoplastic elastomer comprises a Shore hardness in the region from 60 A to 100 A, particularly 70 A to 90 A, particularly 80 A.

Furthermore, according to an embodiment of the capsule, the capsule comprises an ejection mechanism configured to eject the capsule out of a recess of a body of the unit of the apparatus.

The foregoing and other aspects and features of the disclosure will become apparent to those of reasonable skill in the art from the following detailed description, as considered in conjunction with the accompanying drawings.

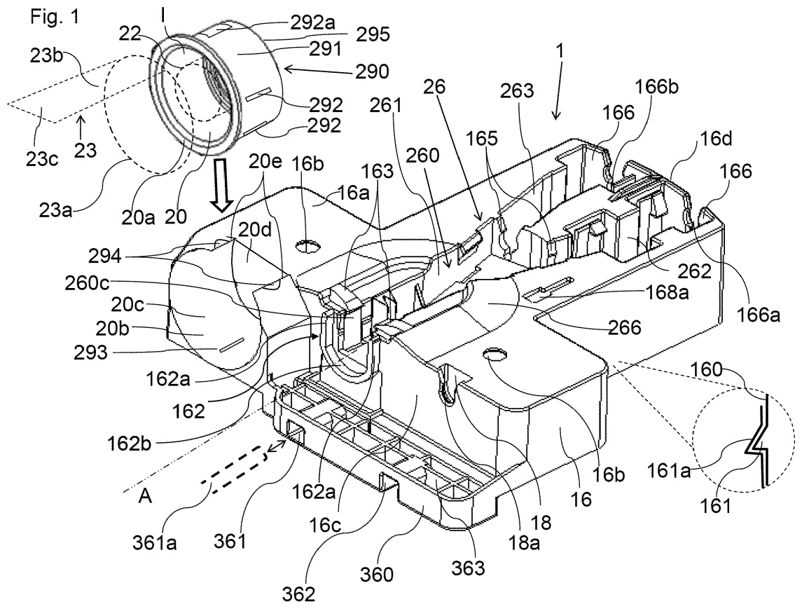

FIG. 1 shows a perspective view of an (e.g. removable) unit according to the invention having a disposable capsule for holding an end cap,

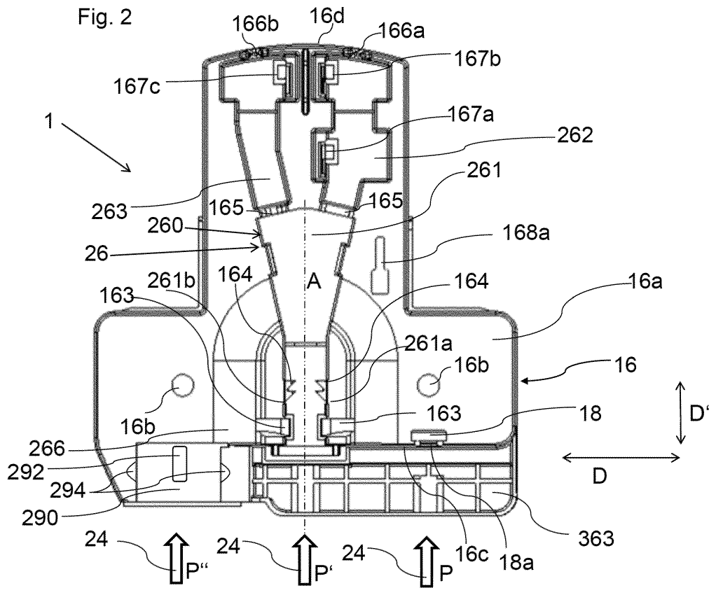

FIG. 2 shows a plan view onto a unit according to the invention,

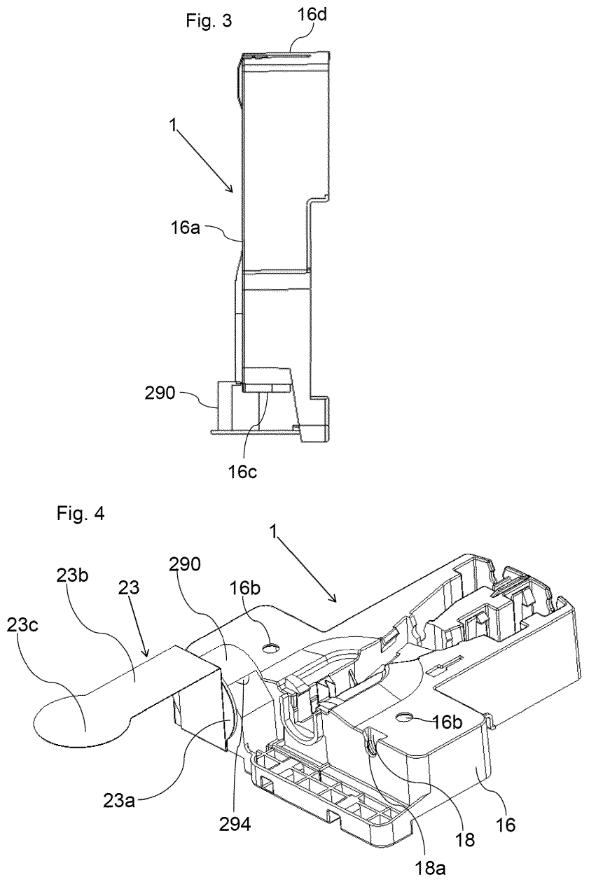

FIG. 3 shows a lateral view onto a unit according to the invention,

FIG. 4 shows a perspective view onto the unit of FIG. 1 wherein the second receptacle is closed with a peel-off seal,

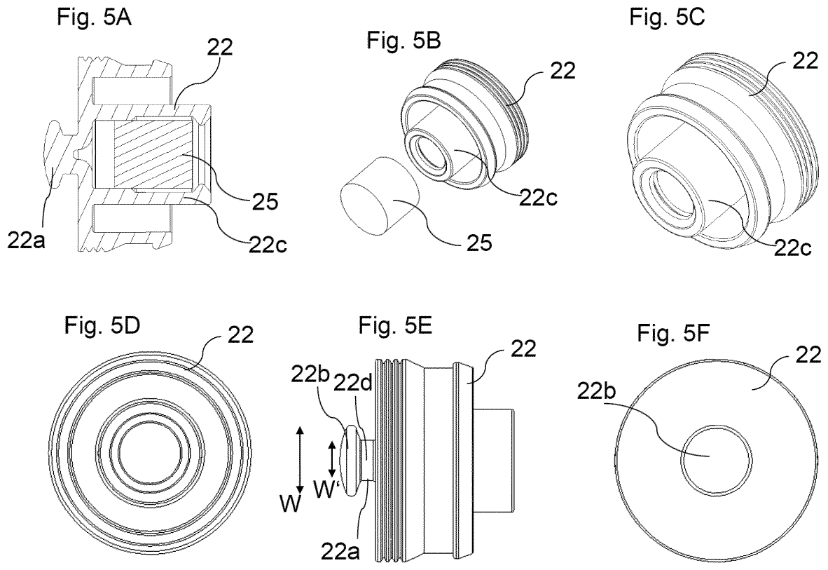

FIG. 5A to 5F show an end cap that can be stored in the second receptacle of the (disposable) capsule,

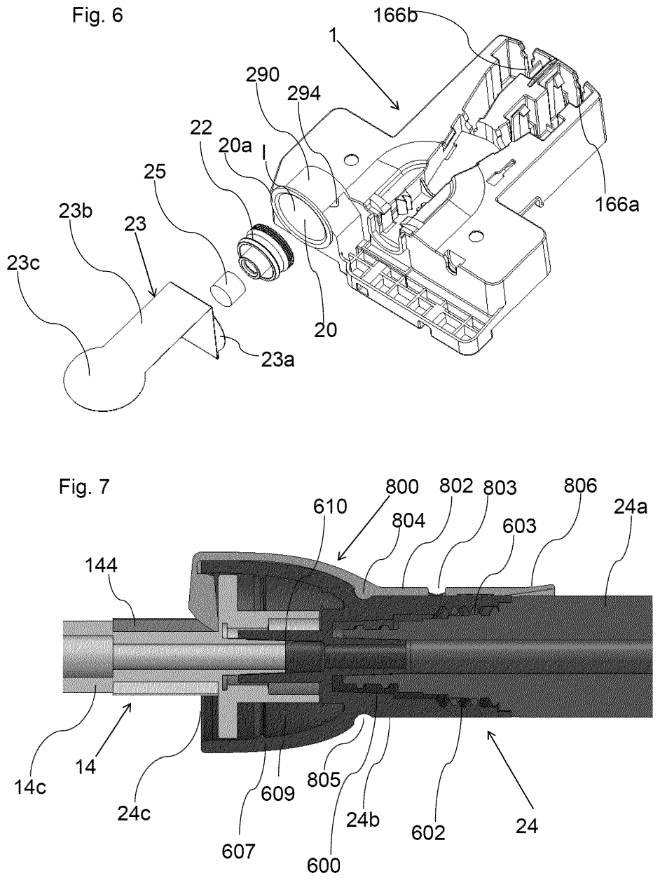

FIG. 6 shows an exploded view of the removable unit of FIG. 4,

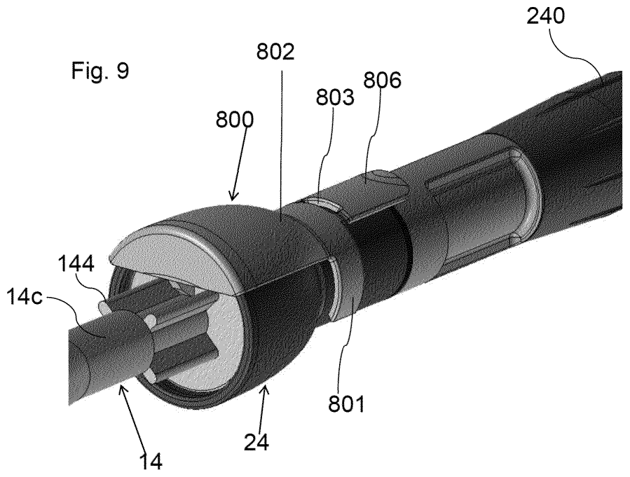

FIGS. 7 to 9 show a tubular fitting according to the invention comprising a removable clamp,

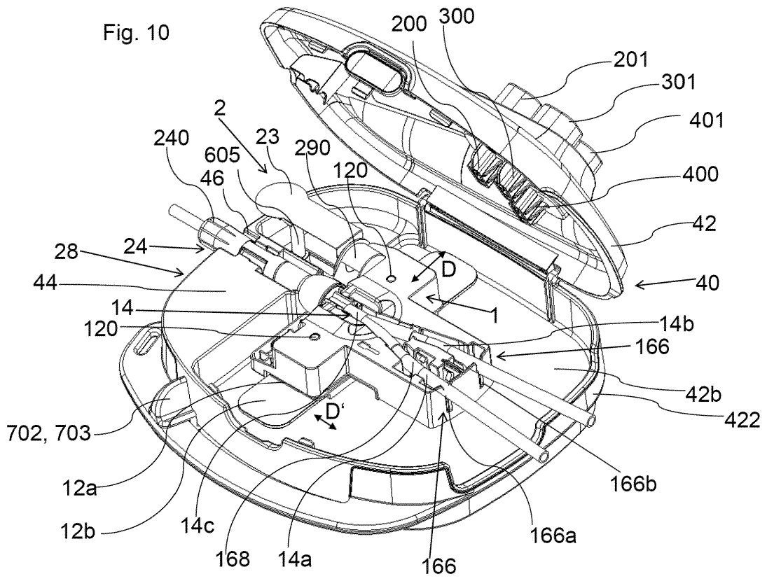

FIG. 10 shows a perspective view of an apparatus for connecting/disconnecting a tubular fitting and a connector that is equipped with a removable unit according to the present invention,

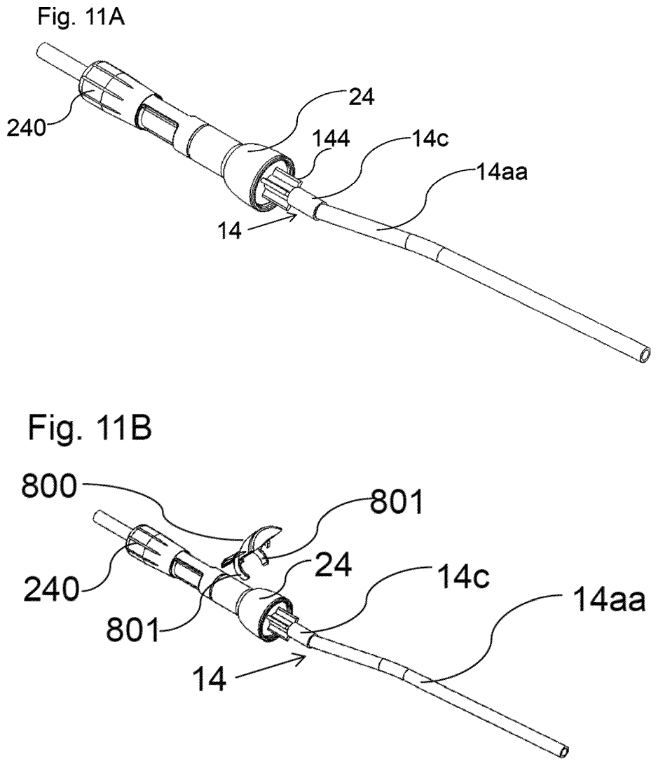

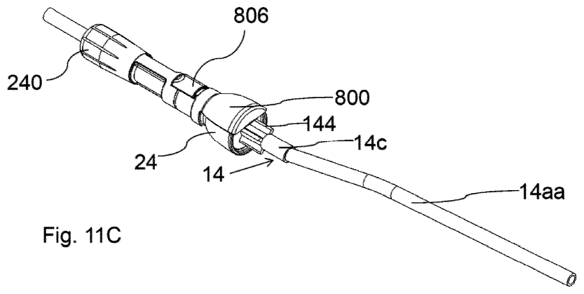

FIG. 11A to 11C show a further connector which comprises only a single conduit (e.g. for APD),

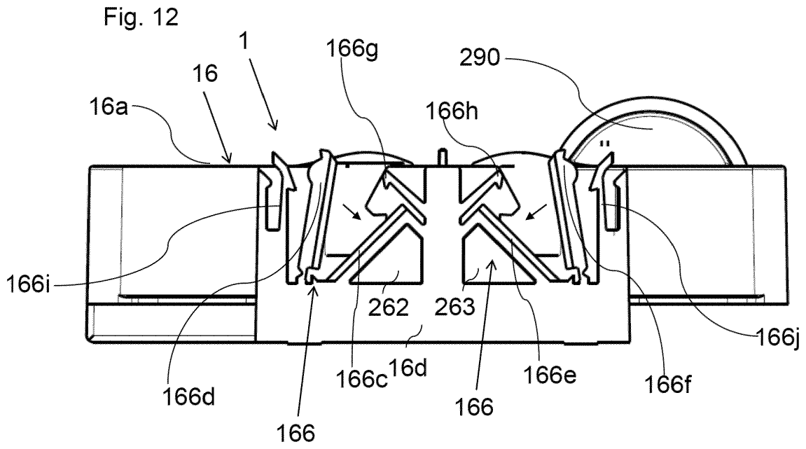

FIG. 12 shows a view of the back side wall of a removable unit according to the present invention with holding means comprising pivotable clamping arms,

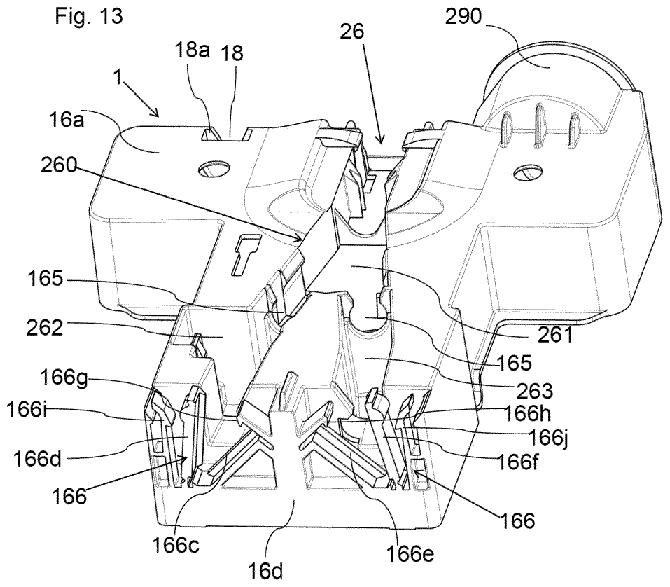

FIG. 13 shows a perspective view of the unit shown in FIG. 12,

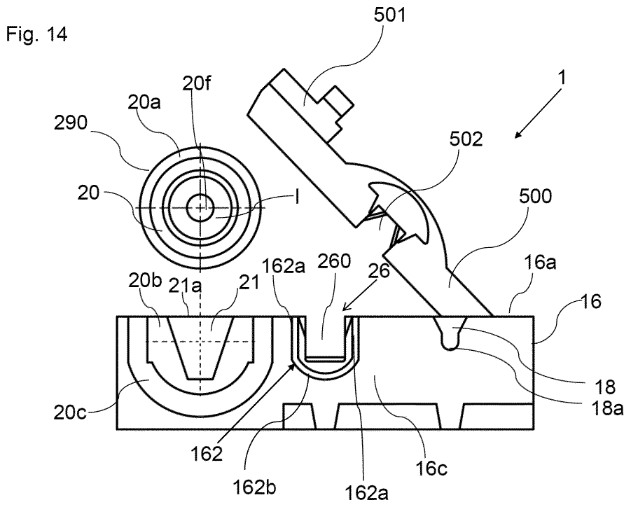

FIGS. 14 to 17 show views of a further embodiment of the unit and capsule according to the present invention, wherein the capsule comprises an axial protrusion that tapers,

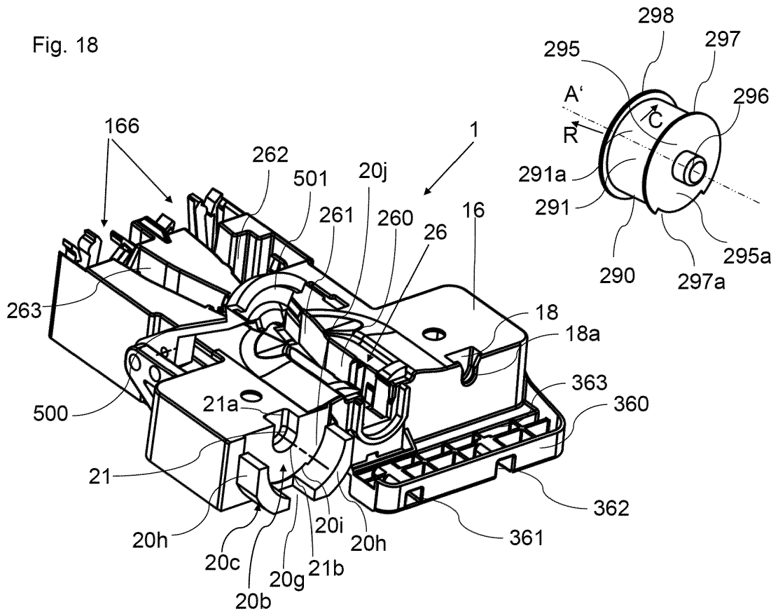

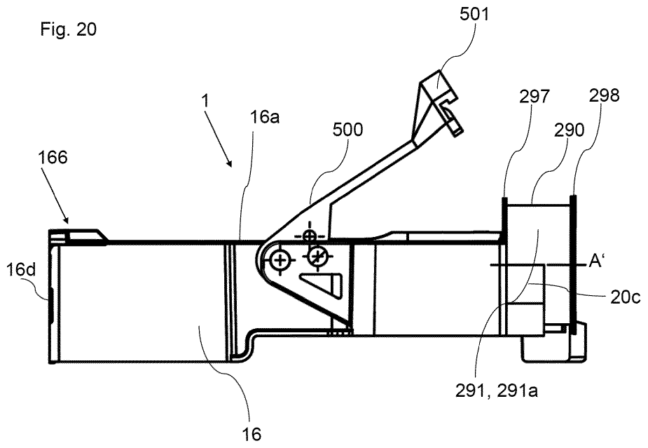

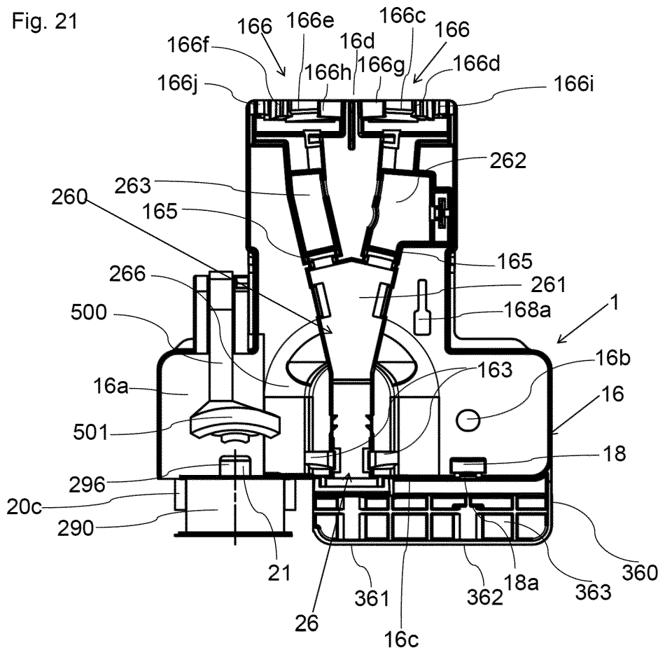

FIGS. 18 to 21 show views of a further embodiment of the unit and capsule according to the present invention,

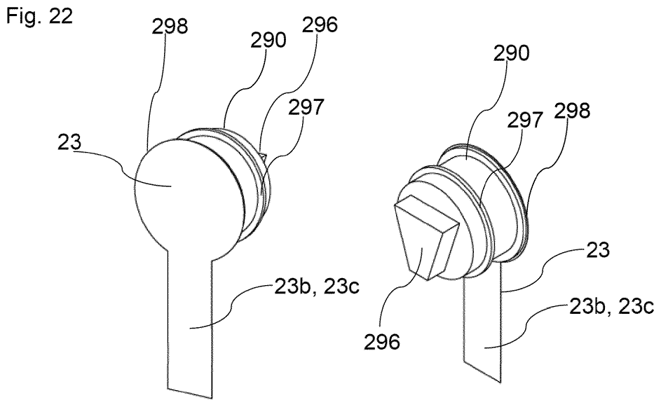

FIG. 22 shows a capsule having an axial protrusion with a closure for enclosing the end cap in the capsule,

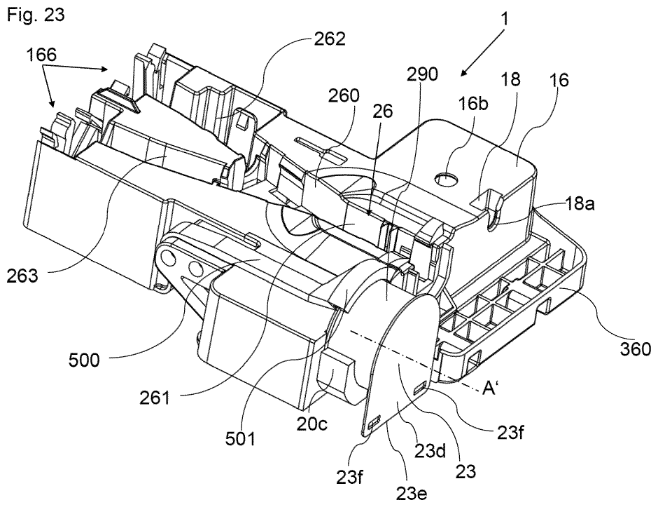

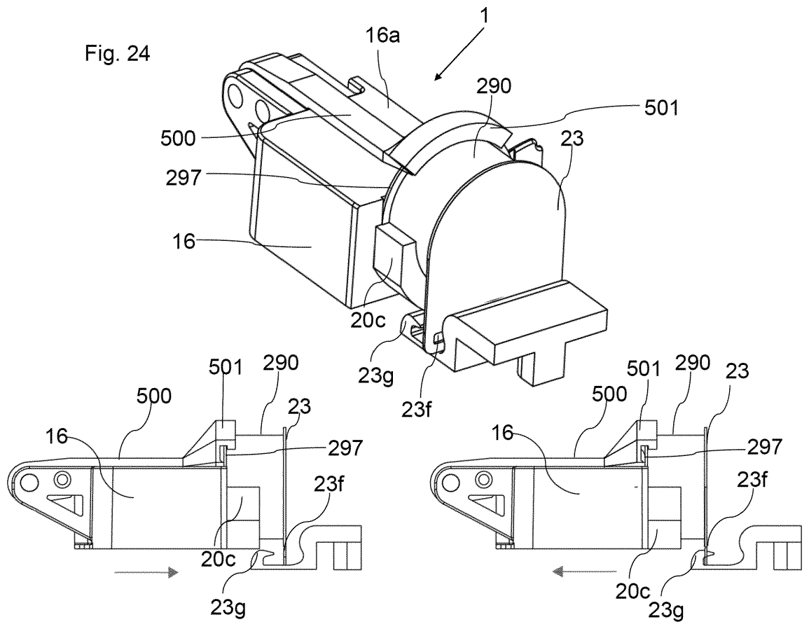

FIGS. 23 to 24 show a modification of the embodiment shown in FIGS. 18 to 21, wherein here the closure is configured to be automatically removed by the apparatus,

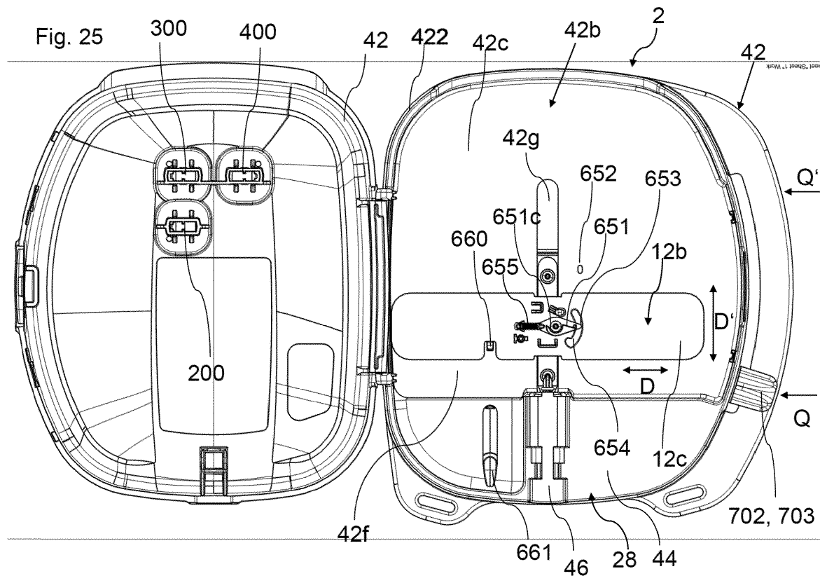

FIG. 25 shows a top view onto an embodiment of an apparatus according to the present invention, wherein the movable carrier is removed from the slider to show the pivotable stopper,

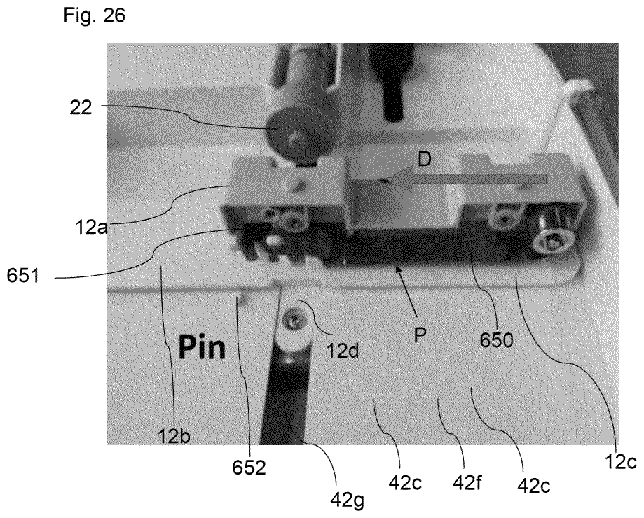

FIGS. 26 to 31 show illustrations of an embodiment of an apparatus according to the present invention (as shown in FIG. 24), wherein the unit is removed from the movable carrier. Further, the movable carrier is shown in an opened configuration to reveal the spring and stopper below the movable carrier,

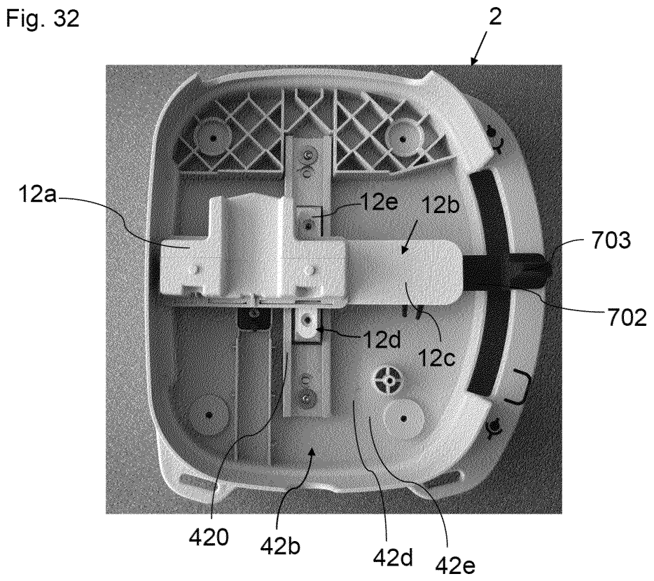

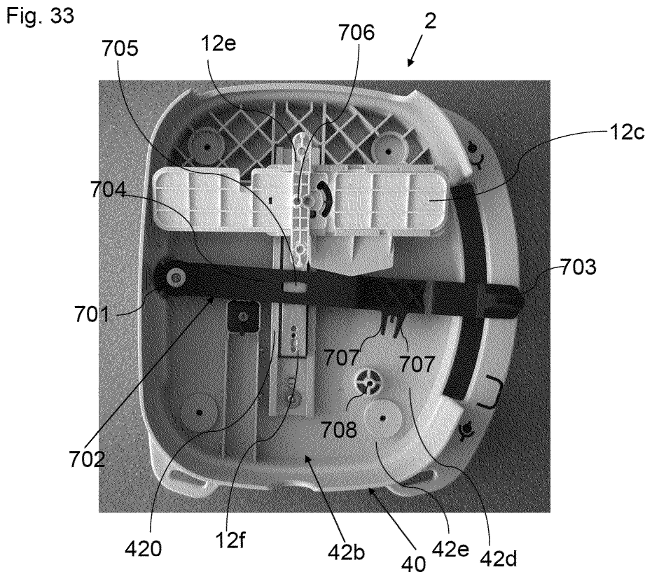

FIGS. 32 to 33 show tops views of the apparatus shown in FIGS. 25 to 31, wherein here a first (upper) bottom wall of the apparatus is removed to show the actuating element (elongated lever) for moving the slider/movable carrier,

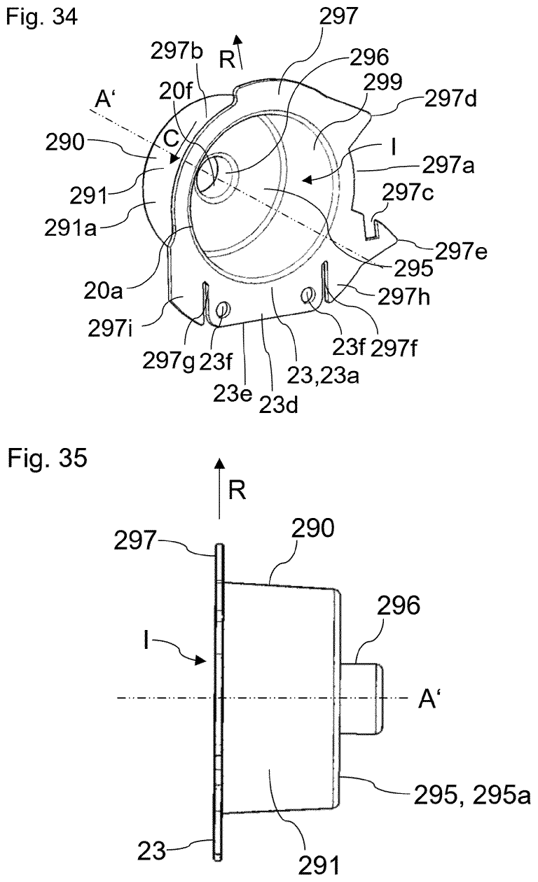

FIGS. 34 to 37 show perspective, side and back views, respectively, of a further embodiment of the capsule according to the invention,

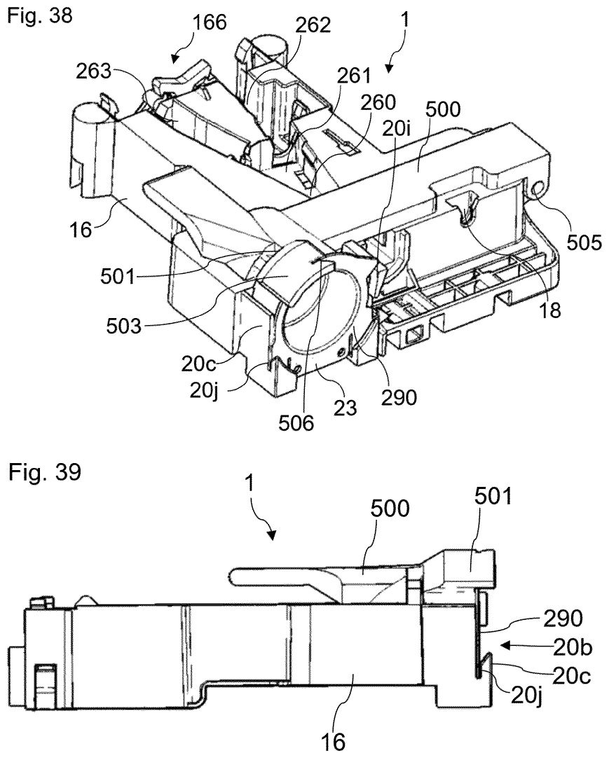

FIGS. 38 to 40 show perspective and side views of a further embodiment of a unit for an apparatus for connecting and disconnecting a tubular fitting to a connector according to the invention, into which a capsule according to FIGS. 34 to 37 can be inserted,

FIG. 41 shows a back view of the unit depicted in FIGS. 38 to 40 to illustrate a further embodiment of a holding means according to the invention for holding and clamping conduits,

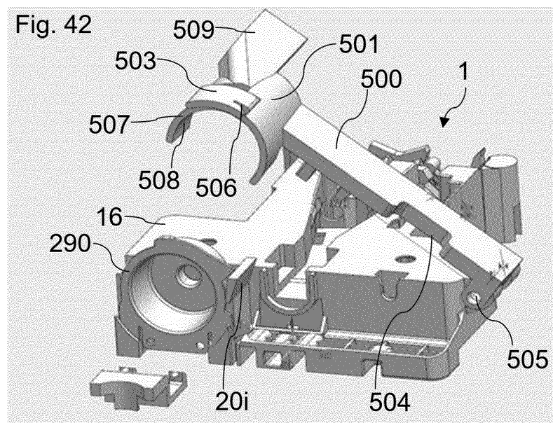

FIG. 42 shows a perspective view of the unit for an apparatus for connecting and disconnecting a tubular fitting to a connector according to the invention with the fastening lever in the first position.

FIG. 1 shows together with FIGS. 2 to 6 an e.g. removable unit 1 for an apparatus 2 for connecting and disconnecting a tubular fitting 24 to a connector 14. An example of such an apparatus 2 in shown in FIG. 10, where the unit 1 is connected to a moveable carrier 12a of the apparatus 2. Also FIGS. 25 to 33 show an apparatus 2 according to the present invention.

According to FIGS. 1 to 3, the preferably disposable unit 1, which can be configured to be discarded after e.g. a single or a finite number of uses comprises a body 16 having a first receptacle 18. Further, the unit 1 also comprises a second receptacle 20, wherein each of said receptacles 18, 20 is configured for receiving or holding an end cap 22 of the tubular fitting 24. Further, the body 16 comprises a connector holder 26 being e.g. disposed between said first and second receptacles 18, 20 or between said first receptacle 18 and the recess 20b/capsule 290 (see below). The connector holder 26 is configured for accommodating insertion of the connector 14.

An embodiment of such an end cap 22 is shown in FIGS. 5A to 5F. Particularly, the end cap 22 comprises a protrusion 22a having a broadened head 22b (cf. FIGS. 5E and 5F), i.e. the diameter W of the head 22b is larger than the diameter W′ of a basis 22d of the protrusion 22a, which basis 22d connects said head 22b to the remaining portion of the end cap 22. Particularly, the head 22b is configured to engage behind an edge 18a of the first receptacle 18 so that the end cap 22 is received in the first receptacle 18 and so that the tubular fitting 24 can be pulled off an end cap 22 when the latter has its head 22b engaged with said edge 18a of the first receptacle 18. Further, particularly, in an embodiment, the end cap 22 comprises an inner protrusion 22c for receiving the protrusion 610 of the tubular fitting 24 (cf. FIGS. 7 to 9 and description below).

Further, FIGS. 7 to 9 show a typical tubular fitting 24 that is used with the unit 1/apparatus 2.

Particularly, in order to remove the unit 1 from the carrier 12a and a surrounding housing 40 comprising a bottom 42b to which the carrier 12a is mounted and an (e.g. transparent) cover 42 in an easy manner, said body 16 of the removable unit 1 (which may also be denoted as a consumable in the framework of the present invention) is configured to be connected to said apparatus 2 in a releasable fashion, e.g. by means of a latching connection that can be manually established or released.

As already indicated above, the unit 1 is particularly designed for a finite number of uses, each use comprising unfastening an end cap 22 from the tubular fitting 24, connecting the connector 14 inserted into the connector holder 26 of the removable unit 1 to the tubular fitting 24, disconnecting the connector 14 from the tubular fitting 24, and fastening an end cap 22 received in an internal space I of the capsule 290 to the tubular fitting 24. These basic steps will be described in more detail below (see also above).

As indicated in FIGS. 1 and 6, the unit 1 comprises an end cap 22 for closing the tubular fitting 14 after use, which end cap 22 replaces an old end cap 22 that has been unfastened from the tubular fitting 24 before. According to FIGS. 1 and 6, the fresh and particularly sterile end cap 22 for replacement is arranged in the second receptacle 20 that is formed by a disposable capsule 290 that can be releasably fastened to the body 16 of the unit 1, wherein said end cap 22 is completely arranged in said second receptacle 20/capsule 290.

Particularly, the body 16 comprises a recess 20b for receiving the capsule 290 in a form fitting manner, wherein the capsule 290 and/or the body 16 are configured to fasten the capsule 290 to the body 16 (e.g. by a latching connection when the capsule 290 is arranged in said recess 20b). Particularly, the recess 20b for receiving the capsule 290 comprises a lateral wall 20c for enclosing the capsule 290 at least partially when the capsule 290 is arranged in said recess 20b. As indicated in FIG. 1, the lateral wall 20c can comprise a discontinuity 20d arranged between two opposing edges 20e of the lateral wall 20c on the top side 16a of the body 16 of the unit 1.

In an embodiment, a recess 294 may formed in each edge 20e, which recesses 294 face each other. These recesses 294 allow for a more easy removal of the capsule 290 from the recess 20b since they particularly increase flexibility of the edges 20e that retain the inserted capsule 290.

Alternatively, an actuating member 294 may be arranged at each edge 20e for unlocking the latching connection between the capsule 290 and the recess 20b/lateral wall 20c when the capsule 290 is received in said recess 20b.

Furthermore, the capsule 290 comprises a lateral wall 291 surrounding an internal space I of the capsule in which the end cap 22 resides. The capsule 290 further comprises a bottom wall 295 connected (e.g. integrally) to the lateral wall 291 of the capsule 290. Particularly, the lateral wall 291 of the capsule 290 comprises at least one latching nose 292 that is configured to engage with an associated latching recess 293 formed in said lateral wall 20c of said recess 20b of the body for receiving the capsule 290. Alternatively, the lateral wall 20c of said recess 20b for receiving the capsule 290 comprises at least one latching nose 293 that is configured to engage with an associated latching recess 292 formed in said lateral wall 291 of the capsule 290. Said latching noses/recesses may form part of the latching connection/mechanism for fastening the capsule 290 in its recess 20b.

Furthermore, as indicated in FIG. 1 for example, the capsule 290 may comprise an indicator 292a that is configured to indicate whether the capsule 290 is unused (e.g. comprises a fresh end cap 22, see also above). Particularly, said indicator 292a is arranged on the lateral wall 291 of the capsule 290 and can be a window or an indicator 292a that undergoes a visually perceivable change when the capsule 290 has been used.

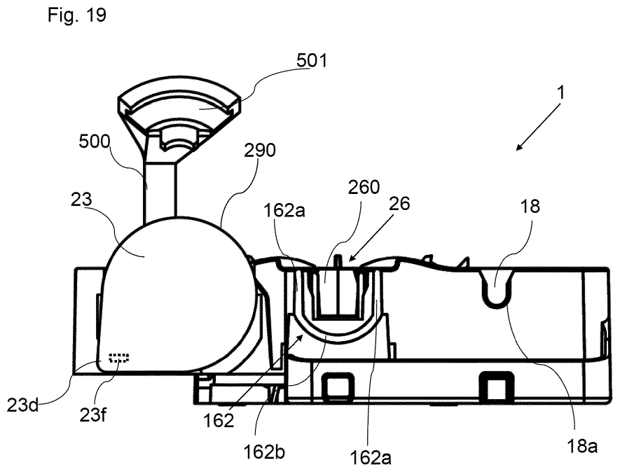

Furthermore, in order to protect the end cap 22 in said capsule 290, the capsule 290 is closed, particularly hermetically closed, by a closure 23 that is shown in FIGS. 1, 4, 6, and 10 (alternative embodiments of the closure 23 are shown in FIGS. 19 and 22 to 24 and will be described further below). Particularly, the closure 23 comprises a peel-off seal (e.g. foil) 23a, which peel-off seal 23a is attached to a circumferential face side 20a of the capsule 290/lateral wall 291 of the capsule 290 (cf. FIG. 1). Further, said closure 23 comprises a flexible strip 23b connected to the peel-off seal 23a, which strip 23b comprises a free end section 23c which may be configured to be arranged between the bottom 42b and the cover 42 of the housing 40 of said apparatus 2 such that the free end section 23c of the strip 23b protrudes out of the housing 40 and thus forms a handle (having e.g. a flat circular shape) by means of which the peel-off seal 23a can be peeled off the capsule 290 from outside said housing 40 by simply pulling on the free end section 23c (e.g. with two fingers).

For further protection of the end cap 22, the unit 1 can comprise a flexible carrier member 25, particularly a sponge 25, that carries an anti-bacterial agent or fluid, and is in contact with said end cap 22 and also arranged in the second receptacle 20 (together with the end cap 22). Particularly, the flexible carrier member 25 can be inserted into the end cap 22 as shown in FIG. 5A, particularly into said inner cylindrical protrusion 22c of the end cap 22.

As already indicated above, the body 16 of the unit 1 is configured to be connected to said movable carrier 12a of said apparatus 2 (cf. FIG. 10) in a releasable fashion, wherein particularly said carrier 12a is movable with respect to the bottom 42b of the housing 40 of the apparatus 2 so that the removable unit 1 can be moved together with said carrier 12a inside said housing 40 in the different positions P, P′, and P″ that will be described in more detail below with reference to FIGS. 2 and 10 as well as FIGS. 24 to 32

Further, the body 16 comprises a top side 16a, wherein two (e.g. circular) through-holes 16b are formed in the body 16 on the top side 16a which are each configured to receive an associated pin 120 that protrudes from said carrier 12a (cf. FIG. 10) when the body 16 is connected to the carrier 12a, wherein particularly a face side of the respective pin 120 is flush with the top side 16a when the removable unit 1 is connected properly to the carrier 12a of the apparatus 2.

Furthermore, in order to have the connector 14 inserted correctly, the top side 16a comprises a through-hole 168a (e.g. having a contour in the form of a frangible inline seal) which indicates the position of a frangible inline seal 168 of a first conduit 14a of the connector 14.

Particularly, for establishing said releasable connection between the body 16 and the carrier 12a, the body 16 comprises two opposing internal surfaces 160 (cf. schematic detail of FIG. 1 in dashed circle) wherein said internal surfaces face each other, and

wherein a latching means 161, particularly a latching nose, is provided on each internal surface for engaging with a complementary latching means, particularly a recess 161a, of the carrier 12a.

Further, the body 16 comprises a front side wall 16c which faces a tubular fitting 24 that shall be connected to the connector 24 or disconnected from the connector 14 and an opposing back side wall 16d (cf. e.g. FIG. 1) via which the conduits 14a, 14b of the connector 14 exit the apparatus 2.

Furthermore, the connector holder 26 comprises a recess 260 (cf. e.g. FIG. 2) for receiving the connector 14 as shown in FIG. 10, which recess 260 is arranged on the top side 16a of the body 16 and extends from the front side wall 16c to the back side wall 16d of the body 16.

Particularly, said recess 260 for receiving the connector 14 comprises an end region 261 extending from the front side wall 16c in the direction of the back side wall 16d, which end region 261 then branches out into a first and a second region 262, 263 of the recess 260 that extend from said end region 261 to the back side wall 16d, respectively.

As can be seen in FIG. 1, at least a portion of the end region 261 of the recess 260 is surrounded by a slight (rounded) protrusion 266 on the top side 16 a on either side of said portion of the end region 261. This protrusion 266 helps the user to place/insert the connector 14 correctly into the recess 260.

Particularly, the Y-shape of the recess 260 allows insertion of a Y-shaped connector 14 (cf. FIG. 10) into the single regions 261, 262, 263 of the recess 260, which connector 14 comprises a first conduit 14a and a second conduit 14b, wherein the two conduits 14a, 14b branch off from an end section 14c of the connector 14, via which end section 14c the connector 14 is configured to be connected to said tubular fitting 24, wherein the end region 261 is configured to receive said end section 14c of the connector 14, and wherein the first region 262 is configured to receive the first conduit 14a, and wherein the second region 263 is configured to receive the second conduit 14b.

Alternatively, as shown in FIGS. 11A to 110, (e.g. in case of APD), the connector comprises only a single conduit 14aa connected to the end section 14c of the connector 14, which single conduit 14aa is configured to be arranged in the second region 263.

For properly holding the connector 14 in the recess 260, the connector holder/recess 260 comprises a plurality of holding means, which will be described in more detail in the following.

Particularly, as shown in FIGS. 1 and 2, the removable unit 1 comprises an e.g. U-shaped protrusion 162 protruding from the front side wall 16c and extending along the end region 261 of the recess 260 on the front side wall 16c, wherein said protrusion 162 is configured to an inclination of the end section 14c of the connector 14 with respect to an axial direction A of the end section 14c when said end section 14c is inserted in said end region 261 of the recess 260, particularly said protrusion 162 comprises two opposing parallel sections 162a that are integrally connected by a lower curved section 162b which parallel sections 162a and curved section 162b are configured to butt against said end section 14c in a form fitting manner to prevent said inclination.

Further, according to an embodiment, the end region 261 of the recess 260 may comprise an (e.g. rectangular) cross sectional contour 260c in a plane perpendicular to said axial direction A, wherein the end section 14c of the connector 14 comprises a shape in the region of said contour 260c so that the connector 14 cannot be rotated about the axial direction A when it is inserted into the recess 260. Particularly said shape of the connector 14 may be formed by four wings 144 that protrude from the end section 14c and engage with said contour 260c of the recess 260.

Further, the body 16 comprises two holding means or parts 163 arranged on the top side 16a of the body 16, wherein the two holding means 163 are arranged on opposing side walls 261a, 261b of the end region 261 of the recess 260 for holding the end section 14c of the connector 14 in place in the end region 261 of the recess 260 when said end section 14c is arranged in said end region 261 of the recess 260.

Particularly, each of said two holding means 163 comprises a free end, wherein the respective free end protrudes past the associated side wall 261a, 261b of the end region 261 of the recess 260, which two side walls 261a, 261b of said end region 261 of the recess 260 face each other. Thus, the fee ends of the holding means 162 can engage over an end section 14c of the connector 14 that is inserted into the end region 261 of the recess 260 to hold the end section 14c in place in the recess 260.

Furthermore, particularly, the body 16 comprises two holding means or parts 164, wherein each of said holding means 164 forms a tooth structure 164, and wherein the respective tooth structure protrudes from an associated sidewall 261a, 261b of the end region 261 of the recess 260, wherein said two sidewalls 261a, 261b, and tooth structures 164 face each other. Thus, the tooth structures 164 can clamp the end section 14c of the connector from both sides and are thus configured to prevent a movement of the end section 14c of the connector 14 in an axial direction A when said end section 14c of the connector 14 is arranged in said end region 261 of the recess 260 and a force pulls said end section 14 in said axial direction A and into the teeth of the tooth structures 164. Other abrasive structures may also be used.

Furthermore, the body 16 comprises a holding means or part 165 arranged in the first region 262 of the recess 262, which holding means 165 is configured to clamp the first conduit 14a of the connector 14 when said first conduit 14a is arranged in the first region 262 of the recess 260 to hold the first conduit 14a in place, particularly without hindering fluid flow through the first conduit 14a. Particularly said holding means 165 comprises a curved edge for tightly encompassing the first conduit 14a.

In the same manner, the body 16 particularly comprises another holding means or part 165 arranged in the second region 263 of the recess 260, which holding means 165 is in turn configured to clamp the second conduit 14b of the connector 14 (or said single conduit 14aa, cf. FIGS. 11A to 11C) when said second conduit 14b is arranged in the second region 263 of the recess 260 to hold the second conduit 14a in place, particularly without hindering fluid flow through the first conduit 14a. Particularly, also here, said holding means 165 comprises a curved edge for tightly encompassing the second conduit 14b.

Furthermore, particularly, the body 16 comprises a holding means or part 166 arranged in the first region 262 of the recess 260 at the back side wall 16d, which holding means 166 is configured to clamp the first conduit 14a of the connector 14 when said first conduit 14a is arranged in the first region 262 of the recess 260, wherein particularly said holding means 166 is configured to encompass said first conduit, e.g. by means of a curved edge for tightly encompassing the first conduit 14a. Further, said holding means 166 comprises a slot 166a extending from said edge into which slot 166a the first conduit 14a can be pushed so that the first conduit 14a is blocked for the passage of fluid through the first conduit 14a.

Likewise, the body 16 further comprises such a holding means or part 166 arranged in the second region 263 of the recess 260 at the back side wall 16d, which holding means 166 is in turn configured to clamp the second conduit 14b of the connector 14 when said second conduit 14b is arranged in the second region 263 of the recess 260, particularly by means of a curved edge for tightly encompassing the second conduit 14b. Also here said holding means 166 of the second region 263 comprises a slot 166b extending from said edge, into which slot the second conduit 14b can be pushed so that the second conduit 14b is blocked for the passage of fluid through the second conduit 14b.

The actuating members 200, 300, 400 (cf. FIG. 10) by means of which the frangible seal 168 can be broken and the conduits 14a, 14b can be blocked will be described in detail below.

FIGS. 12 and 13 show alternative holding means 166 which may be used instead of the holding means 166 comprising said slots 166a and 166b.

Particularly, here, the holding means 166 of the first region 262 of the recess 260 comprises a clamping surface 166c and a pivotable clamping arm 166d, wherein the clamping arm 166d can be pivoted towards the particularly inclined clamping surface 166c into a clamping position so that the first conduit 14a (cf. FIG. 10) is blocked when it is clamped between said clamping surface 166c and the clamping arm 166d. Analogously, the holding means 166 arranged in the second region 263 of the recess 260 at the back side wall 16d, can comprises a clamping surface 166e and a pivotable clamping arm 166f, too, wherein the clamping arm 166f is in turn configured to be pivoted towards the clamping surface 166e into a clamping position so that the second conduit 14b or said at least one conduit 14aa is blocked when it is clamped between said clamping surface 166e and said clamping arm 166f.

Further, the clamping arm 166d of the holding means 166 of the first region 262 of the recess 260 can be configured to be pivoted towards the associated clamping surface 166c by means of the second actuating member 300 of the apparatus (see also below and FIG. 10). Likewise, the clamping arm 166f of the holding means 166 of the second region 263 of the recess 260 can be configured to be pivoted towards the associated clamping surface 166e by means of the third actuating member 400 of the apparatus 2 (see also below and FIG. 10).

Furthermore, the respective holding means 166 may comprise a hook 166g, 166h for engaging with the respective clamping arm 166d, 166f so as to hold the respective clamping arm 166d, 166f in its clamping position. When the respective arm 166d, 166f is engaged with the respective hook 166g, 166g, the respective conduit 14a, 14b, 14aa is clamped between the associated clamping surface 166c, 166e and the associated clamping arm 166d, 166f and is therefore blocked.

Furthermore, each holding means 166 may comprise a guiding arm 166i, 166j for guiding a movement of the respective second or third actuating member 300, 400 upon pivoting the respective clamping arm 166d, 166f towards the associated clamping surface 166c, 166e.

Particularly, in FIG. 13, the capsule 290 may also form an integral part of the body 16 of the unit 1 that cannot be removed from the body 16 (see also above).

Furthermore, the body 16 comprises a first and a second latching nose 167a, 167b at the first region 262 of the recess 260, wherein the respective latching nose 167a, 167b is configured to engage an associated (first and second) actuating member 200, 300 of the apparatus 2 so as to hold the respective actuating member 200, 300 in a pressed position. Particularly the first latching nose 167a is configured to engage with a first actuating member 200 of the apparatus 2 that is configured to break said frangible inline seal 168 of the first conduit 14a and to keep the first conduit 14a open for the passage of fluid, and wherein particularly the second latching nose 167b is configured to engage with a second actuating member 300 of the apparatus 2 that is configured to press the first conduit 14a in said slot 166a.

Furthermore, the body 16 comprises a third latching nose 167c at the second region 263 of the recess 260, wherein said third latching nose 167c is configured to engage an associated third actuating member 400 of the apparatus 2 so as to hold said third actuating member 400 in a pressed position. Particularly, the third latching nose 167c is configured to engage with said third actuating member 400 of the apparatus 2 that is configured to press the second conduit 14b in said slot 166b.

Furthermore, in order to protect the apparatus from spilled fluid from the fluid lines that are connected or disconnected by means of the apparatus 2, the removable unit comprises a drip pan 360 that protrudes from the front side wall 16c of said body 16 and is arranged below the end region 261 of the recess 260 as well as below the first receptacle 18 to receive said leakage fluid. Preferably, said drip pan 360 comprises a plurality of dents 363 for receiving said spilled fluid. Which plurality of dents 363 allows to hold and calm the leakage fluid.

Furthermore, the body 16 may comprise a centering means 361 for guiding a movement of the removable unit 1 in the axial direction A, which centering means 361 is arranged (at least in sections) on the drip pan 360. Particularly, said centering means 361 is formed as a recess 361 of the drip pan 360 for receiving a guiding means, particularly a guiding pin 361a of the apparatus 2, which guiding pin 361a is schematically indicated in FIG. 1).

The drip pan 360 preferably comprises a recess 362 that extends parallel to the recess 361 and allows to arrange the removable unit 1 on the carrier 12a in the first position P (this recess 362 then receives the guiding pin 361a).

Furthermore, in the following, further embodiments of the unit 1 and capsule 290 are described in detail with respect to FIGS. 14 to 33. Particularly, the respective unit 1, particularly the body 16 can also comprise the features described above in relation with the unit 1 according to FIGS. 1 to 13. Particularly, the end cap 22 can be formed as described in conjunction with the FIGS. 5A to 5F.

Particularly, the FIGS. 14 to 17 show embodiments of the unit 1 and the capsule 290. Here, only the front part of the body 16 is shown. However, the body 16 shown in FIGS. 14 to 17 can further comprise the features described above in conjunction with FIGS. 1 to 13.

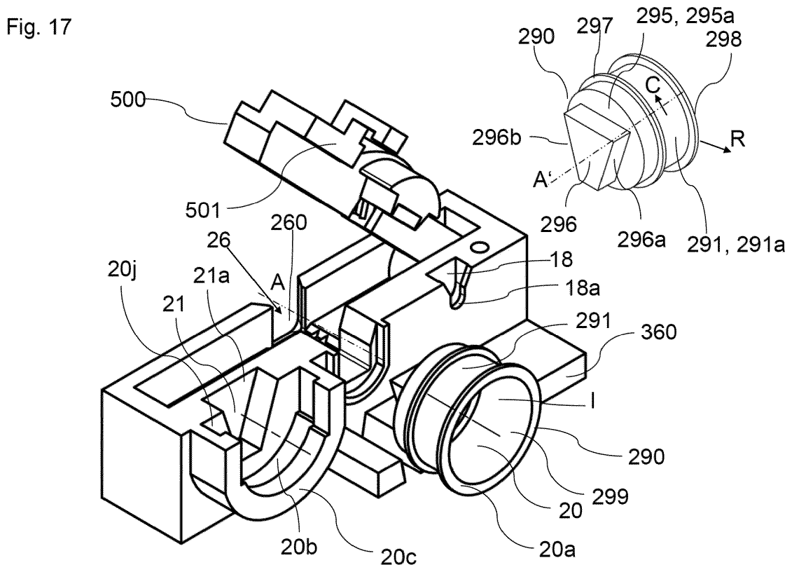

As shown in FIGS. 14 to 17, the capsule 290 comprises a lateral wall 291 surrounding an internal space I of the capsule 290 in a peripheral direction C of the capsule 290, wherein particularly the lateral wall 291 can be cylindrical or can comprise a cylindrical portion. Further, the lateral wall 291 comprises an outside 291a facing away from the internal space I, and particularly an inside facing the end cap 22 residing in the internal space I of the capsule 290. In FIG. 17, the capsule 290 is shown in an opened state (with the closure 23 and end cap 22 removed), but for connecting a fresh end cap 22 to the tubular fitting 24 the capsule 290 enclosed an end cap 22 as described herein (e.g. according to FIGS. 5A to 5F).

The capsule 290 further comprises a bottom wall 295 connected to the lateral wall 291 wherein the lateral wall 291 extends from the bottom wall 295 in an axial direction A′ of the capsule 290 towards a face side 20a of the lateral wall 291, wherein the face side 20a faces away from an outside 295a of the bottom wall 295, which can be circular.

Particularly, the capsule 290 comprises an axial protrusion 296 protruding in the axial direction A′ from the bottom wall 295 on the outside 295a of the bottom wall 295. As can be seen from FIG. 17, the axial protrusion 296 tapers, particularly in a direction perpendicular to the axial direction A′. Particularly, the axial protrusion 296 comprises a planar first surface 296a and a planar second surface 296b, the first surface 296a facing away from the second surface 296b, wherein particularly the first and the second surface 296a, 296b extend perpendicular to an outside 295a of the bottom wall 295 of the capsule 290. The two surfaces 296a, 296b are arranged obliquely with respect to one another causing the axial protrusion 296 to taper as described above.

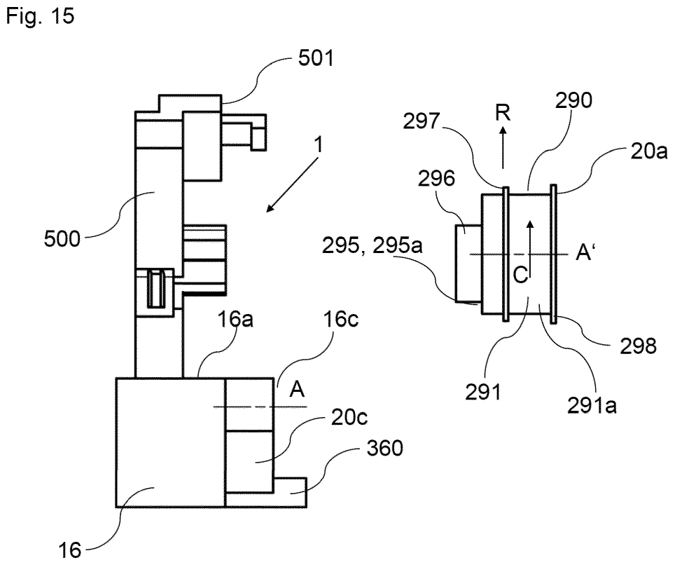

Furthermore, as can e.g. be seen in FIGS. 15 to 17 the capsule 290 can comprises a lateral protrusion 297 protruding in a radial direction R of the capsule 290 from the lateral wall 291 of the capsule 290 on an outside 291a of the lateral wall 291, wherein the lateral protrusion 297 extends in the peripheral direction C of the capsule 290 and is a circumferential (i.e. annular) lateral protrusion 297. The respective radial direction R extends perpendicular to the axial direction A′.

Particularly, the lateral protrusion 297 is spaced apart from the face side 20a as well as from the bottom wall 295 of the lateral wall 291 in the axial direction A′.

Furthermore, the capsule 290 can comprises a further lateral protrusion 298 protruding from the lateral wall 291 on the outside 291a of the lateral wall 291 in the radial direction R of the capsule 290, wherein also the further lateral protrusion 298 extends in the peripheral direction (C) of the capsule (290) and can be a circumferential (i.e. annular) further lateral protrusion 298. Furthermore, the further lateral protrusion 298 is spaced apart from said lateral protrusion 297 in the axial direction A′, and particularly faces said lateral protrusion 297 in the axial direction A′.

Particularly, the further lateral protrusion 298 is arranged adjacent the face side 20a of the lateral wall 295 and is particularly flush with face side 20a of lateral wall 291. As indicated in FIG. 17, the face side 20a delimits an opening 299 of the capsule 290 via which the internal space I of the capsule 290 is accessible, so that the end cap 22 can be removed from the internal space I of the capsule 290. The opening 299 can be closed by means of a closure 23 which will be described in more detail below. Further, also the closure 23, 23a described above can be used with the capsule 290 of FIGS. 14 to 17.

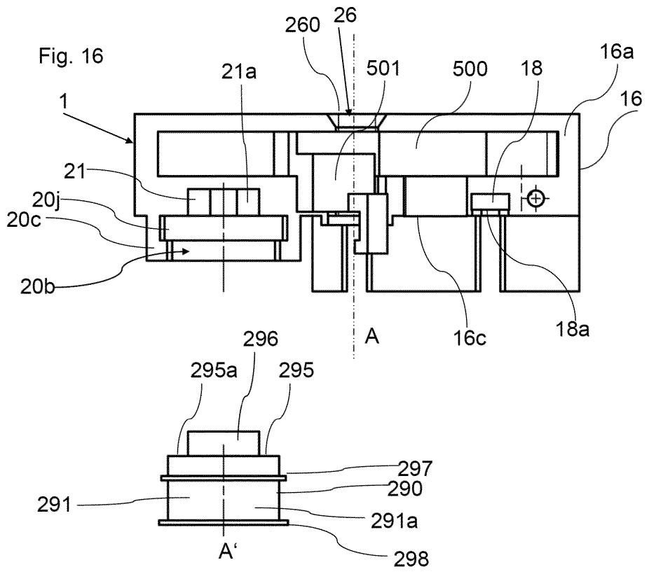

Furthermore, as shown e.g. in FIGS. 16 and 17, also here the unit 1 comprises a body 16 having a recess 20b (e.g. on the front side wall 16c of the body) for receiving the capsule 290. The recess 20b is delimited by a lateral wall 20c for enclosing the capsule 290 at least partially when the capsule 290 is arranged in said recess 20b.

Particularly, said lateral wall 20c of the recess 20b of the body 16 is configured to support the lateral wall 291 of the capsule 290 when the capsule 290 is arranged in the recess 20b of the body 16 and rests on the lateral wall 20c of the recess 20b of the body 16.

As further shown in FIGS. 16 and 17, the recess 20b of the body 16 of the unit 1 comprises a portion 21 that is adapted to the shape of the axial protrusion 296, i.e. the portion 21 is formed as a slot 21 that tapers so that the axial protrusion 296 can be received in the portion 21 of the recess 20b in a form fitting manner. This also fixes an orientation of the capsule 290 in the circumferential direction C of the capsule. Therefore, when the capsule 290 is arranged in the recess 20b a self-positioning of the capsule 290 can be achieved due to the positive fit between the tapered axial protrusion 296 and the tapered slot 21.

Furthermore, as shown in FIGS. 16 and 17, the lateral wall 20c of the recess 20b of the body 16 comprises a groove 20j, wherein said lateral protrusion 297 of the capsule is configured to engage with said groove 20j when the capsule 290 is received in the recess 20b. Particularly, this allows to fix the capsule in the axial direction A′ with respect to the recess 20b/lateral wall 20c.

Furthermore, in order to fasten the capsule to the body 16, the unit 1 can comprise a fastening lever 500 as e.g. shown in FIGS. 14 to 17.

Particularly, the fastening lever 500 is pivotably mounted to the body 16 and is pivotable between a first and a second position, wherein when the fastening lever 500 is in the first position (as e.g. shown in FIG. 17), the fastening lever 500 releases the capsule 290, so that the latter can be removed from the recess 20b of the body 16.

Further, when the fastening lever 500 is in the second position, the fastening lever 500 fastens the capsule 290 to the body 16 in case the capsule 290 is received in the recess 20b of the body 16. In this second or closed position of the fastening lever 500, a portion of the capsule 290 is arranged between the fastening lever 500 and the body 16 (particularly between the fastening lever 500 and the lateral wall 20c of the recess 20b of the body 16) to fasten the capsule 290 to the body 16.

Particularly, when the fastening lever 500 is in the second position, the lateral protrusion 297 of the capsule 290 can engage with said end portion 501 of the fastening lever 500. Further, as can be inferred from FIGS. 16 and 17, when the fastening lever 500 is in the second position with the capsule 290 arranged in the recess 20b (this is achieved in FIGS. 16 and 17 by arranging the capsule 290 in the recess 20b and pivoting the lever 500 downwards) the fastening lever 500 (e.g. said end portion 501) locks the axial protrusion 296 of the capsule 290 that is arranged in said portion 21 of the recess 20b of the body 16 by covering an open side 21a of said portion 21 of the recess 20b (tapers slot 21) of the body 16.

At the same time, when the fastening lever 500 is in the second position it also extends over the recess 260 of the connector holder 26 to secure the connector 14 arranged in the recess 260 of the connector holder 26. Particularly, the fastening lever 500 comprises a recess 502 for receiving the connector 14 when the lever 500 is pivoted to the second position.

Furthermore, FIGS. 18 to 21 show further embodiments of the unit 1 and capsule 290.

Here, in contrast to FIGS. 14 to 17 the axial protrusion 296 comprises a cylindrical shape (cf. e.g. FIG. 18), wherein said portion 21 of the recess 20b of the body 16 forms a slot 21 for receiving this cylindrical axial protrusion 296, wherein the slot 21 comprises a round end 21b particularly adapted to the curvature of the axial protrusion 296 in the peripheral direction of the protrusion 296.

Also here, the capsule 290 comprises a lateral protrusion 297, as shown e.g. in FIG. 18, that protrudes (e.g. in the radial direction R of the capsule 290) from the lateral wall 291 of the capsule 290. Particularly, the lateral protrusion 296 extends along the peripheral direction C of the capsule and comprises a gap 297a. Consequently, to the vicinity of gap 297a, the lateral wall 291 comprises no protrusion. Further, the lateral protrusion 296 can be formed adjacent the bottom wall 295 of the capsule 290, particularly such that the lateral protrusion 297 is flush with the outside 295a of the bottom wall 295.

Particularly, the gap 297a of the lateral protrusion can interact in the following way with the recess 20b of the body 16 as shown in FIG. 18.

Particularly, the lateral wall 20c of the recess 20b of the body 16 comprises two sections 20h separated by a gap 20g, wherein a portion 20i of each section 20h of the lateral wall 20c is configured to engage with the gap 297a of the lateral protrusion 297 of the capsule 290 when the capsule 290 is arranged in the recess 20b. At the same time, the lateral protrusion 297 is received in a groove 20j of each section 20h of the lateral wall 20c. Thus, when the capsule 290 is arranged in the recess 20b of the body 16 of the unit its position with respect to the peripheral direction C of the capsule is fixed since the portions 20i of the lateral wall 20c engage with the gap 297a. Further, since the lateral protrusion 297 is also arranged in said grooves 20j, the capsule is also fixed in the recess 20c with respect to the axial direction A′ of the capsule 290.

As e.g. shown in FIG. 18, the capsule 290 can comprise a further lateral protrusion 298 that can be circumferential (i.e. annular), wherein the further lateral protrusion 298 is particularly formed adjacent the face side 20a of the lateral wall 291 of the capsule 290 and protrudes from the lateral wall 291 in the radial direction R of the capsule 290 while extending annularly in the peripheral direction C of the capsule 290. Consequently, the further lateral protrusion 298 is spaced apart from said lateral protrusion 297 in the axial direction A′ of the capsule 290 and particularly faces said lateral protrusion 297 in the axial direction A′.

Also, according to FIGS. 18 to 21, the body 16 of the unit 1 can comprise a pivotable fastening lever 500 for fastening the capsule 290 to the body 16 when the capsule 290 is arranged in said recess 20b of the body 16, wherein the fastening lever 500 is mounted to the body 16, such that the fastening lever 500 is pivotable between a first and a second position, wherein when the fastening lever 500 is in the first position, the fastening lever 500 releases the capsule 290 (e.g. FIG. 18), and wherein when the fastening lever 500 is in the second position (e.g. FIG. 23), the fastening lever 500 fastens the capsule 290 to the body 16 when the capsule 290 is received in the recess 20b of the body 16. As can be seen from FIG. 23, in the second position of the fastening lever 500, a portion of the capsule 290 is arranged between the fastening lever 500 and the body 16 (particularly between the fastening lever 500 and the lateral wall 20c of the recess 20b of the body 16) to fasten the capsule 290 to the body 16. Further, particularly, the when the fastening lever 500 is in the second position, the lateral protrusion 297 of the capsule 290 engages with the fastening lever 500, particularly with the end portion 501 of the fastening lever 500.

Furthermore, the end portion 21 of the lever 500 can be configured to engage with the portion 21 (e.g. slot 21) of the recess 20b of the body 16 perpendicular to said axial direction A′ of the axial protrusion 296, so that the axial protrusion is locked in the slot 21 in a direction perpendicular to the axial direction A′.

Further, while the lever 500 of FIGS. 14 to 17 is configured to be pivoted in a plane that extends perpendicular to the axial direction A′ of the capsule 290 when the capsule 290 is arranged in the recess 20b of the body 16 of the unit 1, the lever 500 according to FIGS. 18 to 21 is configured to be pivoted in a plane that extends parallel to the axial direction A′ when the capsule 290 is arranged in the recess 20b of the body 16 of the unit 1.

Furthermore, particularly, in the embodiments shown in FIGS. 14 to 21, the axial protrusion 296 of the respective capsule 290 surrounds a portion 20f of the internal space I of the capsule 290 (cf. e.g. FIG. 14), wherein said protrusion 22a of the end cap 22 is arranged in said portion 20f of the internal space I so that particularly the end cap 22 residing in the internal space I comprises a stable position in the internal space I of the capsule 290.

As already described above, the capsules 290 according to the present invention described herein are preferably closed by means of a closure 23 that can comprise a peel-off seal or can be formed as a peel-off seal. It is to be noted that each closure 23 described herein can be combined or used with each of the capsules 290 or units 1.

In the following, closures 23 are described in particular that can be removed automatically. However, such a closure 23 as shown e.g. in FIGS. 23 and 24 can also be replaced by a closure 23 that is removed manually, as shown for instance in FIGS. 19 and 22, although the closure 23 shown in FIG. 19 may optionally comprise a through-opening 23f for interacting with a hook element 23g as will be described further below. The closure 23 shown in FIG. 22 essentially corresponds to the closure 23 described in conjunction with FIGS. 1, 4 and 6 and comprises a flexible strip 32b that forms a free end section 23c that can be used as a handle (e.g. as described above).

For removing the preferably flexible closure 23 automatically, the closure 23 that is preferably attached to the face side 20a of the lateral wall 291 of the respective capsule 290 can comprise at least one through-opening 23f, as e.g. shown in FIGS. 19, 23, and 24.

Particularly, the at least one through-opening 23f is formed in an edge region 23d of the closure 23, wherein particularly said edge region 23d is a free edge region that protrudes from the lateral wall 291 of the capsule 290 (e.g. perpendicular to the axial direction A′).

Particularly, the closure 23 can comprise a further through-opening 23f as shown e.g. in FIGS. 23 and 24, wherein the further through-opening 23f can be formed in said edge region 23d of the closure 23, too.

Particularly, said edge region 23d of the closure 23 comprises a section 23e of an edge of the closure 23, which section 23e extends linearly, wherein the at least one through-opening 23f (and particularly also the further through-opening 23f) is arranged adjacent said section 23e of the edge of the closure 23.

Particularly, the two through-openings 23f can be spaced apart from each other in a direction extending parallel to said section 23e of the edge of the closure 23.

Furthermore, particularly, the two through-openings 23f can be equidistantly spaced apart from said section 23e of the edge in a direction perpendicular to said section 23e of the edge of the closure 23.

In order to remove the closure 23 automatically, the apparatus 2 can comprise a hook element 23g (or two such hook-elements 23g in case there are two through-openings 23f provided in the closure 23).

Particularly, the respective hook-element 23g is arranged such that the edge region 23d can move over the respective hook element 23g when the body 16 of the unit 1 is moved towards a holder assembly 28 of the apparatus 2, which holder assembly is configured to hold the tubular fitting 24 (cf. e.g. lower left portion of FIG. 24) and such that the respective hook element 23g engages with an associated through-opening 23f when the body 16 of the unit 1 is moved away from the holder assembly 28 (cf. e.g. lower right portion of FIG. 24), so that the respective hook-element 23g peels the closure 23 off the face side 20a of the lateral wall 291 of the respective capsule 290. The holder assembly 28 is shown e.g. in FIG. 10 and will be described in more detail below.

Furthermore, FIGS. 7 to 9 show an embodiment of a tubular fitting 24 for use with a removable unit 1/apparatus 2 according to the invention, wherein the tubular fitting 24 is configured to be received by an opening 46 of a body 44 of the holder assembly 28 of said apparatus 2 as indicated e.g. in FIG. 10.

As shown in FIGS. 7 to 9, the tubular fitting 24 comprises a first portion 24a and an adjacent second portion 24b comprising an opening 24c. Particularly, the second portion 24b is configured such that an end cap 22 according to the present invention can be fastened to the second portion 24b by plugging the end cap 22 into said opening 24c of said second portion 24b, and wherein the second portion 24b is configured such that an end cap 22 can be unfastened from the second portion 24b by pulling the end cap 22 out of said opening 24c. Please note that an end cap 22 is not shown in FIGS. 7 to 9, but can replace the connector 14 shown in FIGS. 7 to 9 when the connector is removed from the tubular fitting 24.

Further, particularly, the second portion 24b is configured such that the connector 14 can be connected to the second portion 24b by plugging the connector 14 into said opening 24c of said second portion 24b, and wherein the second portion 24b is configured such that a connector 14 can be disconnected from the second portion 24b by pulling the connector 14 out of said opening 24c.

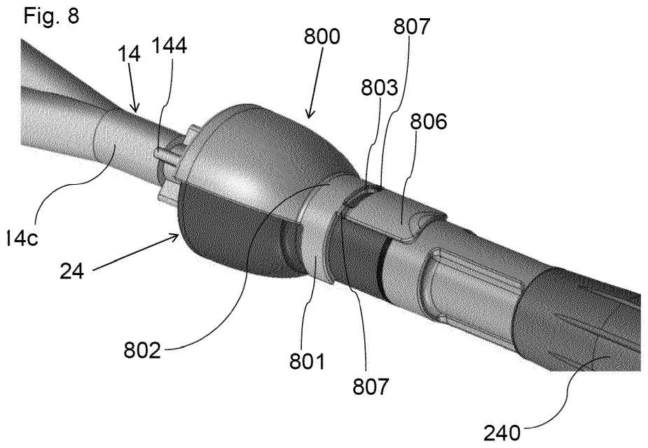

Furthermore, the tubular fitting 24 comprises a removable clamp 800 (cf. e.g. FIGS. 7 and 8) for arresting the connector 14 with respect to the second portion 24b when the connector 14 is connected to the second portion 24b, wherein particularly the clamp 800 is configured to be connected to the second portion 14b, particularly by means of a clip-on connection, wherein particularly the clamp 800 encompasses the second portion 24b when it is connected to the second portion 24b.

For this, the clamp 800 preferably comprises curved arms 801 (cf. also FIGS. 11A to 110) extending from a base 802 of the clamp 800 in a peripheral direction of the second portion 24b for tightly encompassing the second portion 24b which establishes the clip-on connection.

Furthermore, the clipped-on clamp 800 covers a portion of the shroud 607/opening 24c and of said opening 24c when the clamp 800 is connected to the second portion 24b, wherein the clamp 800 is configured to engage behind a portion of the connector 14 when the connector 14 is connected to the second portion 24b so that the connector 14 cannot be disconnected from the second portion 24b.

Furthermore, for defining a location of the clamp 800 on the second portion 24b, the clamp 800 comprises a protrusion 804 for insertion into a circumferential groove 805 of the second portion 24b when the clamp 800 is connected to the second portion 24b.

Further, the clamp 800, comprises a through-hole 803 in a region where the arms 801 project from the base 802, such that a flap 806 is formed that is hinged to the base 802 in a flexible manner (e.g. via integral hinges 807 on either side of the hole 803). This allows to remove the clamp 800 manually from the tubular fitting 24 by lifting the flap 806 away from the fitting 24.

As shown in FIGS. 7 to 9 the second portion 24b is formed as an adapter (or the tubular fitting 24 is formed as an adapter when the first portion 24a is not considered as a component of the tubular fitting/adapter 24, but as a part of a catheter etc.), which comprises a first recess 600 at a first end of the adapter 24b, which first recess 600 comprises an internal thread 602 configured to be rotationally fastened to an external thread 603 of the first portion 24a, and wherein the adapter 24b comprises a shroud 607 at an opposite second end of the adapter 24b, which shroud 607 surrounds a second recess 609 of the adapter 24b into which a protrusion 610 of the adapter 24b protrudes, which protrusion 610 comprises said opening 24c of the adapter 24b such that the shroud 607 surrounds said protrusion 610 and said opening 24c of the adapter 24b, wherein the shroud 607 is coaxially arranged with respect to said protrusion 610.

Alternatively, the first and second portion 24a, 24b may be integrally connected to one another.

FIG. 10 shows the unit 1 when installed into the apparatus 2. As shown in FIG. 10 in conjunction with FIGS. 25 to 33 the apparatus 2 comprises a means, particularly an actuating element 702, for operating the movable carrier 12.

Particularly, said actuating element 702 comprise a handle 703 that is accessible from outside said housing 40 of the apparatus 2 so that the movable carrier 12a is movable (e.g. relative to said holder assembly 28) along a first direction D from a first position P to a second position P′, and from the second position P′ to a third position P″, and particularly from the third position P″ back to the first position P. Particularly, the carrier 12a is reciprocable along said first direction D (cf. FIGS. 1 and 2). These positions P, P′, P″ with respect to the tubular fitting 24 that is inserted into the holder assembly 28 are e.g. indicated in FIG. 2 with arrows.

Particularly, as shown in FIG. 25, the housing 40 can comprise a lateral wall 422 that is connected to the bottom 42b of the housing 40, wherein the cover 42 can be pivotably mounted to said lateral wall 422 such that the cover 42 is pivotable to open or close the housing 40. When the housing 40 is closed, the cover covers the unit 1 and capsule 290. When the housing 40 is open, the unit 1 and/or capsule 290, the tubular fitting 24, and the connector 14 can be removed from the apparatus (particularly from the carrier 12a).

Instead of said actuating element 702 also a drive such as one or several (e.g. electrical) motors may be used for moving the carrier 12a (and particularly slider 12b).

Particularly, the moveable carrier 12a is mounted to a slider 12b (cf. e.g. FIG. 25) so that the carrier 12a is movable along the first direction D with respect to the slider 12a in the respective position P, P′, P″, and wherein the slider 12b is movable (e.g. back and forth) along a second direction D′ which runs perpendicular to the first direction D. Thus, the slider 12b can be moved towards and away from the holder assembly 28.

Further, said holder assembly 28 can comprise a body 44 having an opening 46 for receiving the tubular fitting 24. The tubular fitting 24 may comprise recesses for engaging with the body 44 in the region of said opening 46 of the body 44. Further, particularly when the holder assembly 28 is non-moving, the tubular fitting 24 can be allowed to slide back and forth in the opening 46 in the axial direction A. However, in the embodiment shown in FIGS. 10 and 25 to 33, the tubular fitting 24 cannot move in the axial direction A with respect to the holder assembly 28.

Further, as already mentioned above (cf. e.g. FIG. 2 and FIGS. 25 to 33) the carrier 12a/unit 1 is configured to be movable in each of said positions P, P′, P″ towards and away from the holder assembly 28 by means of the slider 12b (see above), wherein this movement can be generated in particular by pivoting said actuating element 702 manually using handle 703 which will be described in more detail below. Other ways of actuating the slider 12b are also conceivable.

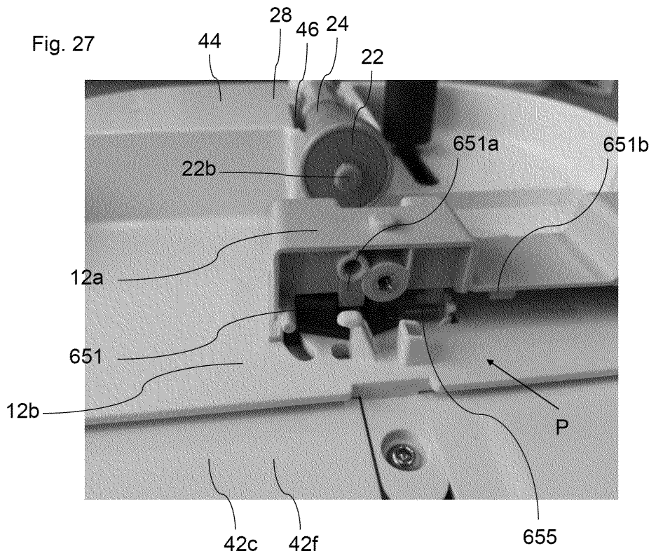

Particularly, when the movable carrier 12a/unit 1 is in the first position P, as indicated in FIG. 2 (cf. also FIGS. 26 and 27), the carrier 12a is movable away from the holder assembly 28 (cf. FIGS. 10 and 25) by means of the actuating element 702, so as to unfasten an end cap 22 received in the first receptacle or port 18 (end cap 22 is fastened to the tubular fitting 24) from said tubular fitting 24, which in turn is inserted into the holder assembly 28, namely in the corresponding opening 46 (cf. FIG. 10).

From there, the movable carrier 12a/unit 1 is moveable into the second position P′ as well as towards the holder assembly 28 by means of the actuating element 702 so as to connect a connector 14 inserted into the connector holder 26/recess 260 to the tubular fitting 24 so that a flow connection can be established between the tubular fitting 24 and the connector 14. When the fluid delivery procedure (which will be described below) is over, the carrier 12a/removable unit 1 is moveable away from the holder assembly 28 by means of the actuating element 702 as indicated by the arrow D′ shown in FIG. 2, so as to disconnect the connector 14 from the tubular fitting 24.

As further shown in FIGS. 10 and 25, the apparatus 2 comprises an openable or removable cover 42 of the housing 40. The cover 42 is preferably at least partially or completely transparent, particularly so as to be able to observe the fastening/unfastening of the end caps 22 and the connecting of the tubular fitting 24 to the connector 14 as well as the disconnecting of the tubular fitting 24 from the connector 14.

Further, the holder assembly 28 may optionally be configured to let the tubular fitting 24 move or slide with the slider 12b/removable unit 1 when the carrier 12a is in the second position P′, such that a member 240 of the tubular fitting 24 protrudes at least partially out of the housing 40 (this member 240 is e.g. shown in FIGS. 8 to 10), e.g. through a through hole 41 formed in the housing 40. Thus, the member 240 is accessible from outside the housing 40 when the tubular fitting 24 is connected to the connector 14 so that the tubular fitting 24 can be opened (e.g. by rotating member 240) and fluid can pass the tubular fitting 24. However, here, particularly, the tubular fitting 24 is non-moving in the axial direction A and said member 240 protrudes out of the housing 40 regardless of the position/movement of the removable unit 1.

Further, as indicated in FIG. 2, when the carrier 12a/unit 1 is in the second position P′ and is moved away from the holder assembly 28, the tubular fitting 24 may optionally also move with the cradle assembly 12 in the beginning so that said member 240 is again positioned inside a compartment defined by the housing 40 and cannot be rotated from outside the housing 40 to ensure that the tubular fitting 24 that has been closed by means of member 240 before disconnecting the tubular fitting 24 from the connector 14 cannot be opened again so that fluid can be lost.

Further, from the second position shown in FIG. 2, the carrier 12a/unit 1 is movable into the third position P″ and towards the holder assembly 28 so that a new end cap 22 that is arranged in the capsule 290 can be fastened to the tubular fitting 24. In this position, the member 240 protrudes again or still out of the housing 40 through opening 41.

Before the new end cap 22 can be fastened to the tubular fitting 24, the closure 23 or peel-off seal 23a described above is removed from the capsule 290 that has been inserted into the recess 20b in beforehand, so that the end cap 22 is accessible for a connection with the tubular fitting 24 (see also above).

Particularly, using the at least one hook element 23g described above, the closure 23 can be automatically removed when the carrier 12a is e.g. in the second position P′ and moved away from the holder assembly 28 to move it in the third position P″ (cf. FIGS. 23 and 24).

Furthermore, for operating the connector 14, the apparatus 2 comprises said recess 260 formed on the top side 16a of the body 16 of the removable unit 12.

When the tubular fitting 24 has been connected to the connector 14 as intended (e.g. FIG. 10), the first actuating member 200 is pressed via a button 201 so that said frangible inline seal 168 is broken.

This allows flushing of the conduits 14a, 14b while the tubular member 24 is still closed. Thus fluid (e.g. from a fluid bag) enters the first conduit 14a, is passed into the second conduit 14b and ends up e.g. in a fluid waste bag connected to the second conduit 14b.

When the conduits 14a, 14b have been flushed, a third actuating member 400 is pressed via a button 401 so as to interrupt the passage of fluid through the second conduit 14b arranged in the second region 262 of the recess 260 of the removable unit 1. To accomplish this, the third actuating member 400 is configured to press the second conduit 14b into the associated slot 166b so that the second conduit 14b is compressed and thereby sealed in the slot 166b. Now, the member 240 is actuated and fluid passes from the first conduit 14a via the tubular fitting 24 to a catheter of the patient.

Finally, to end fluid delivery, the member 240 is actuated to close the tubular fitting 24 and a second actuating member 300 (cf. FIG. 10) is pressed via a button 301 so as to interrupt the passage of fluid through the first conduit 14a being arranged in the first region 262 of the recess 260. Again, to accomplish this, the second actuating member 300 is configured to press the first conduit 14a into the associated slot 166a so that the first conduit 14a is compressed and thereby sealed in the slot 166a.

Now, the connector 14 can be disconnected from the tubular fitting 24 and a new end cap 22 received in the second receptacle 20 can be fastened to the tubular fitting 24 as described above.

Alternatively, as already indicated above, instead of the slots 166a, 166b, the holding means 166 comprising said clamping surfaces 166c, 166e and clamping arms 166d, 166f may be used to block/seal the first, second or at least one conduit 14, 14b, 14aa by actuating the corresponding (second or third) actuating member 300, 400. Here, as already described above, the clamping arm 166d can be pivoted downwards/towards its clamping surface 166c by means of the second actuating member 300 (i.e. by pushing button 301) for sealing the first conduit 14a. Further, the clamping arm 166f can be pivoted downwards/towards its associate clamping surface 166e by means of the third actuating member 400 (i.e. by pushing button 401) for sealing the second conduit 14b or the at least one conduit 14aa.

It is to be noted, that the actuating members 200, 300, 400 are preferably mounted such to the housing 40 or with respect to the recess 260 of the unit 1 that the carrier 12a can move relative to the actuating members 200, 300, 400, but the latter are able to engage with the respective conduit 14a, 14b as described above, when the carrier 12a resides in the second position P′ and has been moved towards the holder assembly 28 so that the connector 14 is properly connected to the tubular fitting 24.

Preferably, the buttons 201, 301, 401 are labeled by means of a pictogram, respectively, so as to describe their specific function to a user. This allows a description of the function of the buttons 201, 301, 401 that is independent of any language and universally understandable. The buttons may also be labeled by means of numbers that indicate the order of operation of the buttons.

Furthermore, according to an embodiment of the apparatus 2, the slider 12b is configured to be moved back and forth along the second direction D′ by means of an actuating element 702 in the form of an elongated lever 702.

Particularly, the slider 12b is mounted to the bottom 42b of the apparatus 2 such that it is movable with respect to the bottom 42b back and forth along the second direction D′.

Particularly, as can be seen from FIGS. 25, 32 to 33, the bottom 42b of the apparatus is a double bottom and comprises a first (e.g. upper) bottom wall 42c and a second (e.g. lower) bottom wall 42d, wherein the bottom 42b comprises an interspace 42e arranged between the first bottom wall 42c and the second bottom wall 42d, and wherein the first bottom wall 42c comprises a top side 42f facing away from the second bottom wall 42d (or from the interspace 42e).

Furthermore, the slider 12b can comprise a first portion 12c arranged on the top side 42f of the first bottom wall 42c, and a second portion 12d connected to the first portion 12c of the slider 12b. Particularly, the carrier 12a is mounted to the first portion 12c of the slider 12b. Further, particularly, the second portion 12d of the slider 12b extends through a slot 42g formed in the first bottom wall 42c into the interspace 42e.

Further, the second portion 12d of the slider 12b (and therewith the slider 12b) is guided by a guide rail 420 along the second direction D′, wherein the guide rail 420 is arranged in the interspace 42e and/or connected to the second bottom wall 42d of the bottom 42b. Particularly, the second portion 12d of the slider 12b can comprise a first part 12e connected (e.g. integrally) to the first portion 12c of the slider 12b. Further, the second portion 12d of the slider 12b can comprise a second part 12f that is connected to the first part 12e (e.g. by screws) and that is guided by the guide rail 420. The second part 12f can be seen in FIG. 33 in which the first bottom wall 42c is removed to allow inspection of the interspace 42e, and wherein the first portion 12c of the slider 12b together with the first part 12e is disconnected from the second part 12f and turned upside down to show the actuating element 702.

As shown in FIG. 33, the actuating element 702 is formed as an elongated lever 702 having a first end section 701 and an opposing second end section 703, wherein the first end section 701 of the actuating element 702 is arranged in the interspace 42e of the bottom 42b and is pivotably mounted to the bottom 42b, particularly to the second bottom wall 42d and/or to the first bottom wall 42c of the apparatus 2. Furthermore, the second end section 703 of the actuating element 702 forms the handle 703 for pivoting the actuating element 702 manually. Particularly, the second end section 703 or handle 703 protrudes out of the housing 40 of the apparatus 2.

Furthermore, the actuating element 702 comprises a connecting section 704 that connects the first end section 701 to the second end section 703 of the actuating element 702. Particularly, the connecting section 704 is coupled to the second portion 12d of the slider 12b such that the slider 12b is movable (e.g. back and forth) along the second direction D′ by pivoting the actuating element 702. Further, for coupling the actuating element 702 to the second portion 12d of the slider 12b, the actuating element 702 comprises an oblong hole 705 in the connecting section 704, wherein the second portion 12d of the slider 12b may comprise an element 706 (e.g. formed by said first part 12e or a portion thereof) that engages with the oblong hole 705 and thus moves the slider 12b along the second direction D′ when the actuating element 702 is pivoted using the handle 703.

Particularly, as shown in FIG. 25, the actuating element 702 is configured to be pivoted back and forth between a first position Q and a second position Q′, wherein when the actuating element is pivoted (e.g. from the second position Q′) towards the first position Q, the slider 12b moves towards the holder assembly 28 and when the actuating element is pivoted (e.g. from the first position Q) towards the second position Q′, the slider 12b moves away from the holder assembly 28.

Furthermore, as shown in FIG. 33, the actuating element 702 comprises two latching arms 707 protruding from the connecting section 704, wherein the apparatus 2 can further comprise a member 708 arranged in the interspace 42e (member 708 can be connected to the first and/or second bottom wall 42c, 42d), wherein said member 708 forms a stop for the actuating element 702 (e.g. when the actuating element 702 resides in the first position Q), and wherein particularly said member 708 is configured to engage with the latching arms 707 (e.g. to arrest the actuating element 702) when the actuating element 702 is arranged in the first position Q.

Furthermore, according to an embodiment, the actuating element 702 is also configured to move the movable carrier 12a along the first direction D with the help of a spring 650, which is shown in FIGS. 25 to 31.

As can be seen from FIG. 26 the spring 650 connects the carrier 12a which is slidably arranged on the slider 12b, particularly on the first portion 12c of the slider 12b, to the slider 12b, namely to said first portion 12c of the slider 12b.

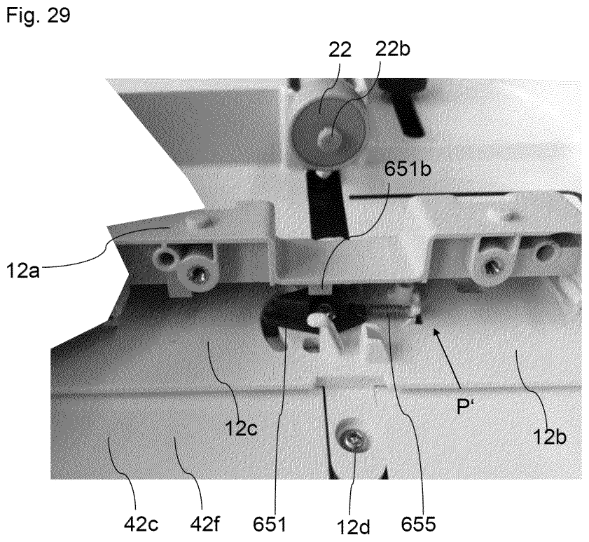

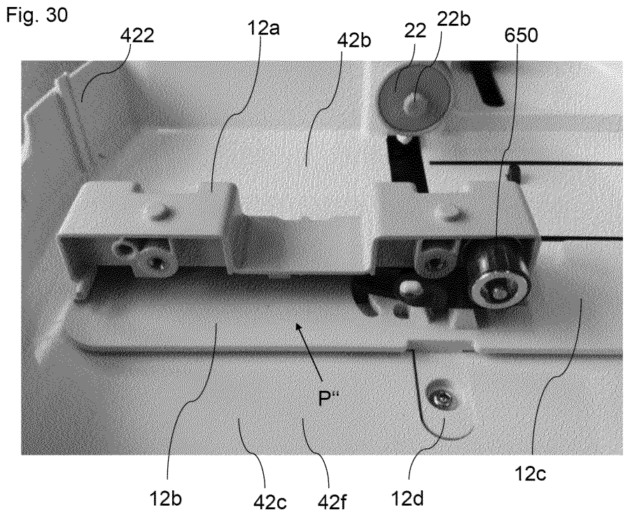

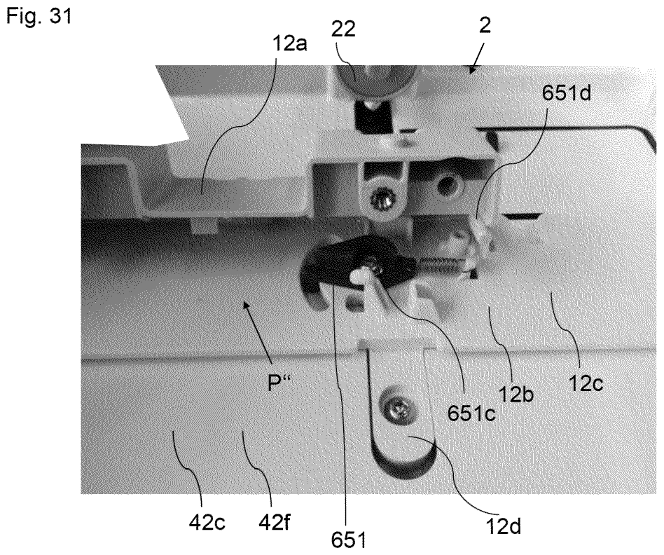

When the spring 650 is fully unwound, the carrier 12a is pretensioned in the first direction D and will move with respect to the slider 12b in the first direction D from the first position P′ shown in FIGS. 26 to 28 to the second position P′ shown in FIG. 29 and thereafter into the third position P″ shown in FIGS. 30 and 31 due to a force exerted by the spring 650.

Particularly, in FIG. 26, a front of the carrier 12a (the carrier 12a carries the disposable unit 1) is removed so that one can see the spring 650. The spring 650 can be tensioned by moving the carrier 12a manually into the first position P shown in FIGS. 26 and 27, wherein in FIG. 27 the spring 650 has been removed in order to show the pivotable stopper 651 that is also shown in FIG. 25.

As shown in FIG. 25, the stopper 651 is pivotably mounted to the first portion 12c of the slider 12b and is connected via a spring element 655 to the first portion 12c of the slider 12b, wherein the spring element 655 is configured to exert a restoring force on the stopper 651 for pivoting the stopper 651 back into a stopping position in which the stopper 651 stops the carrier 12a (e.g. in the first and second position P, P′).

Furthermore, the stopper 651 comprises an end portion 653 that extends through a curved slot 654 formed in the first portion 12c of the slider 12b. Particularly, the end portion 653 of the stopper 651 is configured to contact the pin 652 when the slider 12 is moved along the second direction D′. When the slider 12b is moved away from the holder assembly 28 the stopper 651 can be pivoted such that it releases the carrier 12a.

Returning now to FIG. 27, one can see that the pivotable stopper 651 stops the carrier 12a in the first position P by blocking a first projection 651a of the carrier 12a.

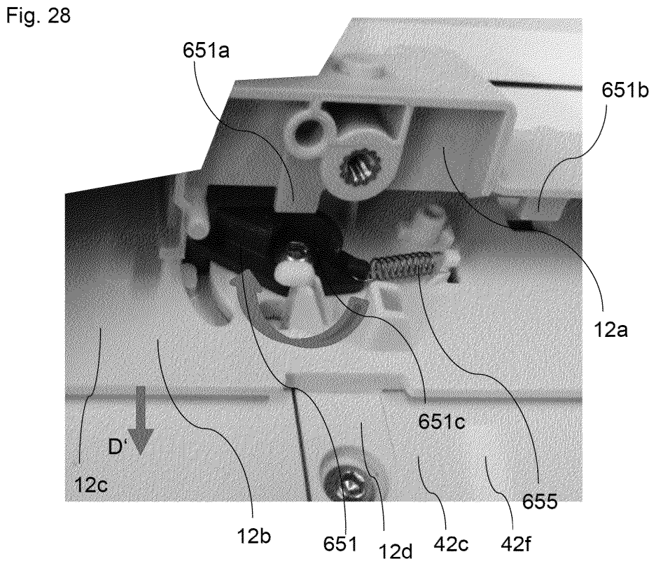

Particularly, the stopper 651 comprises a recess 651c for receiving the projection 651a which is formed such that the first projection 651a is released when the slider 12b is moved away from the holder assembly 28 in the second direction D′ which is shown in FIG. 28. The spring 651 then moves the carrier 12a into the second position P′, where a second projection 651b of the carrier 12a falls into the recess 651c of the stopper 651 so that the carrier 12b is stopped again. This is shown in FIG. 29.

Moving the slider 12b away from the holder assembly 28 again by means of the actuating element 702 releases the second projection 651b and there is a third hard stop in form of the wall 651d of the carrier 12a that stops the carrier 12a in the third position P″ shown in FIGS. 30 and 31. Now, the spring 650 is completely wound up. Moving the carrier 12a (e.g. manually) back into the first position P along the first direction tensions the spring 650 again.

Furthermore, as shown e.g. in FIG. 25, the apparatus 2 can comprise a locking mechanism 660 configured to prevent movement of the slider 12b when a protective cap (not shown) closing the connector 14 has not been connected to a movable locking bar 661 (for this the protective cap can comprise a loop that can be put over the locking bar 661) of the mechanism 660. Particularly, the locking mechanism 660 can be released to allow moving the slider 12b when the locking bar 661 of the apparatus 2 is moved from a first position of the locking bar 661 (shown in FIG. 25) to a second position of the locking bar 661. Particularly, the locking bar 661 is pretensioned towards the first position and is configured to be held in the second position by laying said loop of the protective cap around the locking bar 661. In this way, when the carrier member 12a is moved away from the holder assembly 28 in the first position P of the carrier 12a, the protective cap can also be pulled off the connector 14 by means of the locking bar 661.

Most preferably, in the framework of the present invention as described herein, the connecting and/or disconnecting of connector 14 and tubular fitting 24 (as well as recapping) is performed by one of: a patient using the apparatus at home (e.g. a home dialysis patient), by a caregiver using the apparatus at the patient's home or within a health care facility, or by a healthcare professional using the apparatus at the patient's home or in a health care facility.

A further embodiment of the capsule 290 according to the invention and the corresponding unit 1 for an apparatus 2 for connecting and disconnecting a tubular fitting 24 to a connector 14, in which the capsule 290 can be inserted, is described hereafter with reference to FIGS. 34 to 40 and FIG. 42.

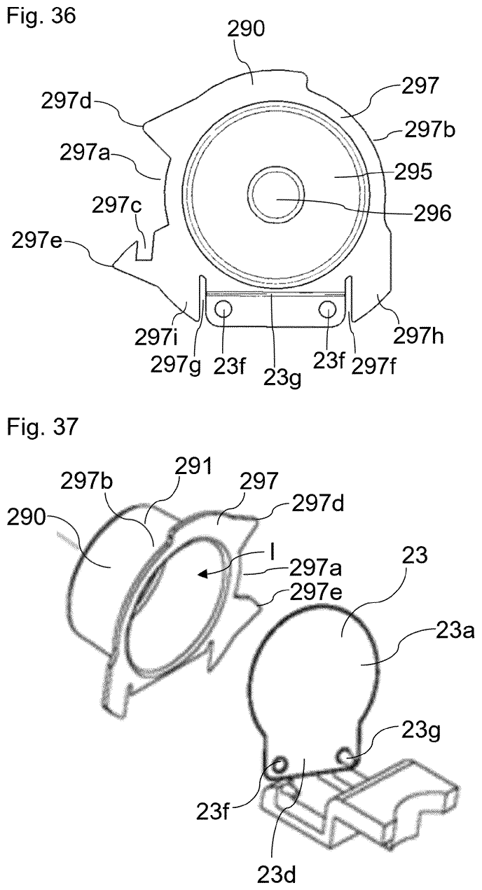

FIG. 34 shows a perspective view of an embodiment of the capsule 290, which is particularly manufactured from a single piece of a thin sheet like material, facilitating the manufacturing process. A side view of the same embodiment is depicted in FIG. 35, and FIG. 36 shows a back view of the capsule 290 viewed from the bottom wall 295.

The capsule 290 comprises an essentially cylindrical portion with a central cylinder axis A′. A circular bottom wall 295 of the capsule 290 extends perpendicular to the axis A′ on an end face of the cylindrical portion. The bottom wall 295 comprises an axial protrusion 296 protruding in the axial direction A′ from the bottom wall 295 on an outside 295a of the bottom wall, wherein the axial protrusion 296 is arranged in the center of the bottom wall 295. Furthermore, the capsule 290 comprises a lateral wall 291 extending in the axial direction from the bottom wall 295 towards a face side 20a of the capsule 290 along the axis A′.

At the face side 20a, the capsule 290 comprises an opening 299 into an internal space I enclosed by the bottom wall 295 and the lateral wall 291. The capsule 290 is configured to receive in the internal space I an end cap 22 of a tubular fitting 24 as shown, for instance, in FIG. 5A to 5F, and optionally also a flexible carrier member 25, such as a sponge, carrying an antibacterial agent or fluid. The axial protrusion 296 defines a portion 20f of the internal space I. When an end cap 22 of a tubular fitting 24 is arranged in the internal space I of the capsule 290, the axial protrusion 296 is configured to surround a protrusion 22a of the end cap 22 (see FIGS. 5a to 5f).

The capsule 290 further comprises a lateral protrusion 297 extending from the face side 20a in the peripheral direction C and in a radial direction R perpendicular to the axial direction A′. The lateral protrusion 297 comprises a first gap 297a and a second gap 297b arranged around the periphery of the lateral protrusion 297. A third gap 297c extending essentially along the peripheral direction C branches off from the first gap 297a or is adjacent to the first gap 297a. On opposite sides of the first gap 297a along the peripheral direction C, the lateral protrusion 297 forms a first radial projection 297d and a second radial projection 297e. The second radial projection 297e is arranged adjacent to the third gap 297c and forms a hook and/or is shaped as an arrow pointing in the radial direction R essentially perpendicular to the extension of the third gap 297c. Adjacent to the second radial projection 297e, the lateral protrusion 297 comprises a first flap 297h, and adjacent to the second gap 297b, the lateral protrusion 297 forms a second flap 297i (delimited on the inside, respectively, by the first slot 297f and the second slot 297g described below).

Furthermore, the capsule 290 comprises a closure 23, comprising a peal-off seal 23a, which is particularly configured to hermetically seal the opening 299 leading into the interior space I of the capsule 290, in particular to keep a sterile environment prior to use of the end cap 22 contained in the capsule 290. The closure 23 covers the entire opening 299, as well as at least a part of the exterior surface of the lateral protrusion 297. In particular, the closure 23 covers an edge region 23d of the lateral protrusion 297 with an edge 23e extending in the peripheral direction C at the bottom of the capsule 290.

The edge region 23d comprises through-openings 23f extending in the axial direction A′, as well as a first slot 297f between the respective through-opening 23f and the first flap 297h, and a second slot 297g between the respective through-opening 23f and the second flap 297i, wherein the first slot 297f and the second slot 297g both extend perpendicular to the axial direction. The through-openings 23f are arranged between the slots 297g along the peripheral direction C.

FIG. 37 illustrates how the closure 23 can be automatically peeled off from the capsule 290 using a movable part of the apparatus 2 (see e.g. FIG. 10) comprising hooks 23g. The movable part is moved, particularly below the capsule 290, along the axial direction A′ in the direction from the bottom wall 295 of the capsule 290 towards the face side 20a, such that the two hooks 23g engage into the through-holes 23f and pull the closure 23 from the capsule 290, thereby opening the internal space I. Such an automatic peel-off mechanism avoids accidental contamination of the internal space I, which may occur during manual handling.

The edge region 23d of the capsule 290 may be formed entirely by the closure 23 (particularly the peel-off seal 23a) or the edge region 23d may comprise an inner layer formed by a part of the lateral protrusion 297 of the capsule 290 and an outer layer formed by the closure 23 (particularly the peel-off seal). In the latter case, the through-openings 23f extend through both the inner layer and the outer layer, and the inner layer (the lateral protrusion 297) may comprise a cut 23g extending parallel to the edge 23e between the opening 299 and the through-openings 23f (see FIG. 36). By means of such a cut 23g, a portion of the inner layer can be removed from the capsule 290 along with the closure 23 when the closure 23 is automatically peeled off by the hooks 23g of the apparatus 2. The closure 23 may consist of any suitable material, which may be particularly transparent or opaque. In particular, the closure 23 shown in FIG. 34 may be from a transparent material.

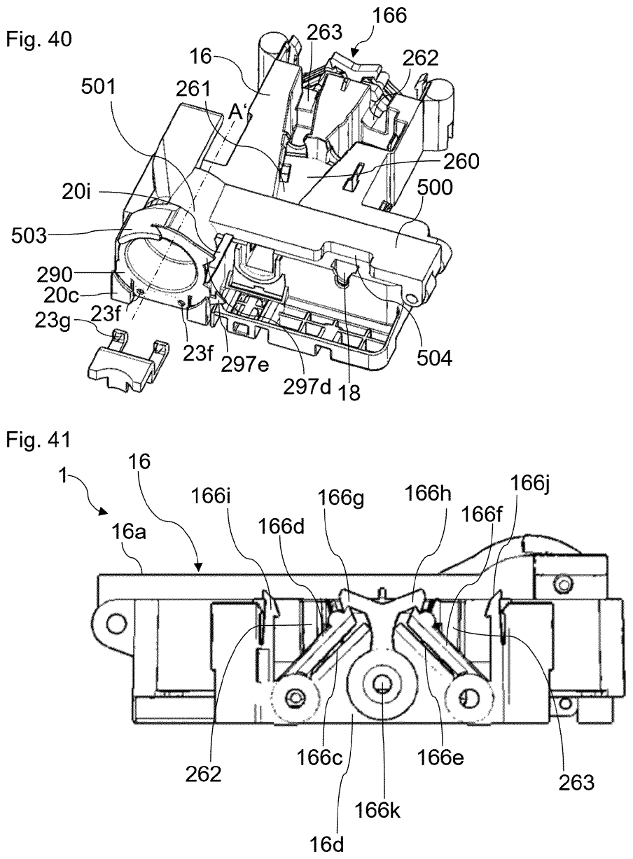

FIGS. 38 to 42 show different views of a unit 1 comprising a capsule 290 according to FIG. 34 to FIG. 37. Other aspects of the unit 1 except those described below may be identical or similar to those of the unit 1 described above and depicted in particular in FIG. 17 and FIG. 18. FIGS. 38 and 39 are perspective views of the unit 1 and FIG. 40 shows a side view of the unit 1.

The unit 1 comprises a body 16 comprising a first receptacle 18 as described above and a recess 20b, in which the capsule 290 is positioned, particularly in a form-fitting manner. The recess 20b comprises a lateral wall 20c contacting and engaging the lateral wall 291 of the capsule 290. Therein, a portion 20i of the lateral wall 20c engages into the first gap 297a of the lateral protrusion 297 of the capsule 290 and engages the first radial projection 297d (see FIG. 34). Furthermore, as best seen in FIG. 40, the second radial projection 297e points towards the front opening of the recess 260 of the unit 1 in a direction perpendicular to the axis A′.

As illustrated in the side view of FIG. 39, the first flap 297h and the second flap 297i of the lateral protrusion 297 of the capsule 290 engage into respective grooves 20j of the lateral wall 20c, which tightly fixes the capsule 290 in the axial direction along the axis A′. This maintains the correct positioning of the capsule 290 also when strong forces are applied in the axial direction, such as e.g. during automatic removal of the closure 23 (see FIG. 37). As best seen in FIGS. 34 and 36, the first flap 297h and the second flap 297i taper towards the respective first slot 297f and second slot 297g, which improves positioning, such that the capsule 290 can easily engage into the grooves 20j.

As best seen in FIG. 38-40 and FIG. 42, the unit 1 further comprises a fastening lever 500 which is pivotably coupled to the body 16 at a pivoting axis 505 which is parallel to the axis A′ of the capsule 290 when the capsule 290 is inserted into the recess 20b. The fastening lever 500 can be pivoted between a first position (shown in FIG. 42) and a second position (FIGS. 38 to 41), where the capsule 290 is engaged by an end portion 501 of the fastening lever 500. The end portion 501 comprises an extension 503 extending in the axial direction of the capsule 290. The extension 503 comprises a connecting means 506, such as a slit for inserting a portion of the lateral protrusion 297 of the capsule 290 to connect the capsule 290 to the lever. In addition, the extension 503 comprises a curved inner surface configured to engage into the second gap 297b of the lateral protrusion 297 of the capsule 290, particularly in a form-fitting manner, thereby tightly fixing the capsule 290 between the lateral wall 20c, the portion 20i and the extension 503. Furthermore, as shown in FIG. 42, the end portion 501 of the fastening lever 500 comprises a curved portion 507 with a concave inner surface 580 which is configured to engage the lateral wall 291 of the capsule 290 (see FIG. 34) in the second position. The fastening lever 500 further comprises a recess 504 which is adjacent to the first receptacle 18 in the second position of the fastening lever 500, such that the first receptacle 18 is accessible for insertion of a protrusion 22a of an end cap 22. For manually moving the lever 500 between the first position and the second position, a handle 509 is arranged at the lever 500, particularly at the end portion 501.

In particular, to insert the capsule 290 into the recess 20b of the unit 1, the capsule 290 may be mechanically connected to the end portion 501 of the fastening lever 500, particularly to the extension 503, and inserted into the recess 20b by moving the fastening lever 500 downwards from the first position (FIG. 42) to the second position (see FIGS. 38 and 40). Fixing of the capsule 290 to the end portion 501 of the fastening lever 500 may for example be achieved by inserting a portion of the lateral protrusion 297 into a connecting means 506 arranged on the end portion 501, particularly on the extension 503. In the example shown in FIG. 38 and FIG. 42, the connecting means 506 is formed as a slit extending in the peripheral direction C in respect of the axis A′ (see FIG. 34), wherein the slit is configured to receive a part of the lateral protrusion 297, particularly between the first gap 297a and the second gap 297b.

During the downward movement of the fastening lever 500 along with the connected capsule 290, the portion 20i of the lateral wall 20c of the unit 1 engages the first radial projection 297d, resulting in particular in a counter clockwise rotation (when viewed from the opening 299, see FIG. 34) of the capsule 290 around the axis A′ in respect of the body 16 of the unit 1. Upon this rotation, the first flap 297i and the second flap 297h engage into the respective grooves 20j (see FIGS. 38 and 39), thereby tightly positioning and fixing the capsule 290 in the axial direction A′. Of course, a similar rotational movement can be achieved by manual insertion of the capsule 290 into the recess 20b.

In particular, to remove the capsule 290 from the unit 1, the fastening lever 500 can be moved upwards from the second position back to the first position along with the connected capsule 290, wherein particularly the capsule 290 is rotated clockwise when viewed from the opening 299. Consequently, the capsule 290 is moved upwards in respect of the body 16 of the unit 1, such that the portion 20i of the lateral wall 20c engages into the third gap 297c, removing the capsule 290 from the fastening lever 500.

It should be noted that the configuration shown in FIG. 42, where the fastening lever 500 is in the first position and the capsule 290 is arranged in the recess 20b of the body 16, usually does not occur during normal operation as described above, since the capsule 290 is normally attached to the fastening lever 500 in the first position.

As shown in FIG. 40, the lateral wall 20c comprises a gap at the bottom adjacent to the edge region 23d of the capsule, such that a tool comprising hooks 23g may be moved along the bottom of the capsule 290, such that the hooks 23g engage into the through-openings 23f to automatically remove the closure 23, particularly peel-off seal 23a, from the capsule 290.

FIG. 41 illustrates a further embodiment of the holding means 166 arranged on or adjacent to the back side wall 16d of the body 16. Of course, this embodiment cannot only be applied to the unit 1 shown in FIGS. 37 to 40, but also to other embodiments of the unit 1, particularly the embodiments depicted in FIGS. 17 and 18.

Similarly to the holding means 166 shown in FIGS. 12 and 13 and described above, the holding means 166 shown in FIG. 41 comprises pivotable clamping arms 166d, 166f, wherein a first conduit 14a provided in the first region 262 of the recess 260 and a second conduit 14b provided in the second region 263 of the recess 260, can be clamped between the respective clamping arm 166d, 166f and corresponding clamping surfaces 166c, 166e. The holding means 166 further comprises guiding arms 166i, 166j for guiding the actuating members 300, 400 of the apparatus 2, respectively, as described above.

In the embodiment shown in FIG. 41, the hooks 166g, 166h for engaging and holding the clamping arms 166d, 166f in the clamping position are coupled to a release mechanism 166k, for example a pressable button. When the release mechanism 166k is activated, the hooks 166g, 166h are displaced, particularly along the axial direction A′ (compare FIG. 40) such that they no longer engage the clamping arms 166d, 166f, and the clamping arms 166d, 166f pivot upwards from the clamping position, thereby releasing the first conduit 14a and the second conduit 14b. Of course, the unit 1 may be adapted such that each clamping arm 166d, 166f is coupled to a separate release mechanism 166k, such that the conduits 14a, 14b can be released individually.

1. A unit (1), particularly for use with an apparatus (2) configured to connect a tubular fitting (24) to a connector (14) and to disconnect the tubular fitting (24) from the connector (14), the unit (1) comprising:
a body (16) having a first receptacle (18) and a connector holder (26) configured to hold the connector (14),
a second receptacle (20), wherein each of said receptacles (18, 20) is configured for holding an end cap (22) of the tubular fitting (24),
wherein the unit (1) further comprises a capsule (290) that is configured to be connected to said body (16) in a releasable fashion, wherein said second receptacle (20) is formed by the capsule (290).

2. The unit according to claim 1, characterized in that the body (16) comprises a recess (20b) for receiving the capsule (290), particularly in a form fitting manner.

3. The unit according to claim 1, characterized in that the recess (20b) of the body (16) for receiving the capsule (290) comprises a lateral wall (20c) for enclosing the capsule (290) at least partially when the capsule (290) is arranged in said recess (20b).

4. The unit according to claim 1, characterized in that the capsule (290) comprises a lateral wall (291) surrounding an internal space (I) of the capsule (290) in a peripheral direction (C) of the capsule (290), and wherein the capsule (290) comprises a bottom wall (295) connected to the lateral wall (291), wherein the lateral wall (291) extends from the bottom wall (295) in an axial direction (A′) of the capsule (290) towards a face side (20a) of the lateral wall (291), wherein the face side (20a) faces away from an outside (295a) of the bottom wall (295).

5.-30. (canceled)

31. The unit according to claim 4, characterized in that the capsule (290) comprises a closure (23) configured to close said internal space (I) of the capsule (290), particularly hermetically, wherein the closure (23) is or comprises a peel-off seal (23, 23a), and wherein the closure (23, 23a) is attached to the face side (20a) of the lateral wall (291) of the capsule (290).

32. (canceled)

33. (canceled)

34. The unit according to claim 31, characterized in that the closure comprises (23) a through-opening (23f), wherein the through-opening (230 is formed in an edge region (23d) of the closure (23), wherein particularly said edge region (23d) is a free edge region, and wherein particularly the edge region (23d) protrudes from the lateral wall (291) of the capsule (290) perpendicular to the axial direction (A′) of the capsule (290).

35.-38. (canceled)

39. The unit according to claim 1, characterized in that the unit (1) comprises a pivotable fastening lever (500) for fastening the capsule (290) to the body (16) when the capsule (290) is received in said recess (20b) of the body (16) of the unit (1), wherein the fastening lever (500) is mounted to the body (16) of the unit (1).

40. The unit according to claim 39, characterized in that the fastening lever (500) is pivotable between a first and a second position, wherein when the fastening lever (500) is in the first position, the fastening lever (500) releases the capsule (290), and wherein when the fastening lever (500) is in the second position, the fastening lever (500) fastens the capsule (290) to the body (16) when the capsule (290) is received in the recess (20b) of the body (16), wherein particularly in the second position of the fastening lever (500) a portion of the capsule (290) is arranged between the fastening lever (500) and the body (16) to fasten the capsule (290) to the body (16), wherein particularly said fastening lever (500) comprises a curved portion (507) comprising a concave inner surface (508) configured to engage said lateral wall (291) of the capsule (290) in the second position.

41.-82. (canceled)

83. An apparatus (2) configured to connect a tubular fitting (24) to a connector (14) and to disconnect the tubular fitting (24) from the connector (14), wherein the apparatus (2) comprises a unit (1) according to claim 1, and a holder assembly (28) configured to hold the tubular fitting (24), wherein the unit (1) is connected or connectable to a movable carrier (12a) of the apparatus (2), and wherein the carrier (12a) is movable with respect to the holder assembly (28).

84.-88. (canceled)

89. The apparatus according to claim 83, characterized in that the apparatus (2) comprises a hook element (23g) configured to engage with the through-opening (23f) of the closure (23) for removing the closure (23) from the capsule (290) when the carrier (12a) is in the second position (P′) and moved away from the holder assembly (28) in the second direction (D′), and wherein particularly the apparatus (2) comprises a further hook element (23g) configured to engage with the further through-opening (230 of the closure (23) for removing the closure (23) from the capsule (290) when the carrier (12a) is in the second position (P′) and moved away from the holder assembly (28) in the second direction (D′).

90.-112. (canceled)

113. A capsule (290), particularly for use with a unit (1) of an apparatus (2) that is configured to connect a tubular fitting (24) to a connector (14) and to disconnect the tubular fitting (24) from the connector (14), wherein the capsule (290) comprises an internal space (I) and an end cap (22) arranged in said internal space (I), wherein particularly the capsule (290) is configured to be connected to a body (16) of the unit (1) in a releasable fashion.

114. The capsule according to claim 113, characterized in that the capsule (290) comprises a lateral wall (291) surrounding said internal space (I) of the capsule (290) in a peripheral direction (C) of the capsule (290), wherein the lateral wall (291) of the capsule (290) comprises an outside (291a) facing away from the internal space (I), and wherein the capsule (290) comprises a bottom wall (295) connected to the lateral wall (291), wherein the lateral wall (291) extends from the bottom wall (295) in an axial direction (A′) of the capsule (290) towards a face side (20a) of the lateral wall (291), and wherein particularly the face side (20a) faces away from an outside (295a) of the bottom wall (295).

115. (canceled)

116. The capsule according to claim 114, characterized in that the capsule (290) comprises an axial protrusion (296) protruding in the axial direction (A′) of the capsule (290) from the bottom wall (295) of the capsule (290) on the outside (295a) of the bottom wall (295), wherein particularly the axial protrusion (296) is configured to engage with a portion (21) of a recess (20b) of the body (16) of the unit (1), particularly in a form fitting manner, wherein the axial protrusion (296) is configured to engage with said portion (21) of the recess (20b) of the body (16) so that a self-positioning of the capsule (290) occurs when the axial protrusion (296) engages with said portion (21) of the recess (20b) of the body (16) of the unit (1)

117.-122. (canceled)

123. The capsule according to claim 114, characterized in that the capsule (290) comprises a lateral protrusion (297) protruding from the lateral wall (291) of the capsule (290) on an outside (291a) of the lateral wall (291), wherein the lateral protrusion (297) extends in the peripheral direction (C) of the capsule (290).

124.-127. (canceled)

128. The capsule according to claim 123, characterized in that the lateral protrusion (297) is a circumferential lateral protrusion, wherein the lateral protrusion (297) comprises at least one gap (297a)

129. (canceled)

130. The capsule according to claim 123, characterized in that the lateral protrusion (297) comprises a first gap (297a), a second gap (297b) and a third gap (297c), wherein particularly said third gap (297c) branches off said first gap (297a) in the peripheral direction (C) of the capsule (290).

131. (canceled)

132. The capsule according to claim 130, characterized in that the lateral protrusion (297) comprises a first radial projection (297d) adjacent to said first gap (297a), and a second radial projection (297e) adjacent to said first gap (297a), wherein particularly said second radial projection (297e) is arranged adjacent said third gap (297c), wherein particularly said second radial projection (297e) forms a hook and/or is shaped as an arrow pointing away from the capsule (290)

133. (canceled)

134. The capsule according to claim 114, characterized in that the capsule (290) comprises a further lateral protrusion (298) protruding from the lateral wall (291) on the outside (291a) of the lateral wall (291) of the capsule (290), wherein the further lateral protrusion (298) extends in the peripheral direction (C) of the capsule (290).

135.-137. (canceled)

138. The capsule according to claim 113, characterized in that said internal space (I) of the capsule (290) is closed, particularly hermetically closed, by a closure (23) attached to the circumferential face side (20a) of the lateral wall (291) of the capsule (290), wherein the closure (23) is or comprises a peel-off seal (23a; 23), and wherein the closure (23) is attached to the face side (20a) of the lateral wall (291) of the capsule (290).

139. (canceled)

140. (canceled)

141. The capsule according to claim 138, characterized in that the closure (23) comprises a through-opening (23f), wherein the through-opening (230 is formed in an edge region (23d) of the closure (23), wherein particularly said edge region (23d) is a free edge region, and wherein particularly the edge region (23d) protrudes from the lateral wall (291) of the capsule (290) perpendicular to the axial direction (A′) of the capsule (290), and wherein the closure (23) comprises a further through-opening (23f), wherein the further through-opening (230 is formed in said edge region (23d) of the closure (23).

142. (canceled)

143. Capsule according to claim 141, characterized in that said edge region (23d) comprises a section (23e) of an edge of the closure (23), which section (23e) preferably extends linearly, wherein the through-opening (23f) is arranged adjacent said section (23e) of the edge of the closure (23), and wherein particularly said further through-opening (23f) is arranged adjacent said section (23e) of the edge, and wherein said closure (23) comprises a first slot (297f) adjacent to said through-opening (23f) and a second slot (297g) adjacent to said further through-opening (23f), wherein said through-opening (23f) and said further through-opening (23f) are arranged between said first slot (297f) and said second slot (297g) in the peripheral direction (C), and wherein particularly said first slot (297f) and said second slot (297g) extend perpendicular to said section (23e) of said edge of said closure (23).

144. (canceled)

145. (canceled)

146. The capsule according to claim 113, characterized in that the capsule (290) comprises an indicator (292a) that is configured to indicate whether the capsule (290) is unused.

147.-152. (canceled)
